(19) United States
(12) Patent Application Publication
(10) Pub. No.: US 2003/0054985 A1
(43) Pub. Date: Mar. 20, 2003

(54) N-cadherin modulated migration, invasion, and metastasis

(76) Inventors: Stuart Aaronson, New York, NY (US); Rachel Hazan, New York, NY (US)

Correspondence Address: BAKER BOTTS L.L.P., 44TH FLOOR, 30 ROCKEFELLER PLAZA, NEW YORK, NY, 10112-4498, US

(21) Appl. No.: 09/790,284

(22) Filed: Feb. 22, 2001

Related U.S. Application Data
(60) Provisional application No. 60/183,852, filed on Feb. 22, 2000.

(51) Int. Cl. A61K 38/18; G01N 33/567

(52) U.S. Cl. 514/12; 435/7.21

(57) ABSTRACT

The present invention relates to methods for modulating the migratory, invasive and metastatic properties of cells expressing N-cadherin for the treatment of proliferative disorders including, but not limited to, cancers such as melanomas, breast, and prostate cancer. The invention further relates to drug screening assays designed to identify compounds that modulate N-cadherin activity and the use of such compounds in the treatment of disorders involving N-cadherin modulated cell migration, invasion, and metastasis. The invention also relates to methods for diagnosis and prognosis of disorders such as cancer that rely on detection of N-cadherin expression levels. The invention is based on the discovery that N-cadherin expression increases the migratory, invasive and metastatic properties of cells. It is additionally based on the discovery that increased N-cadherin expression sensitizes cells to growth factors such as FGF-2 and increases matrix metalloproteinase-9 (MMP-9) accumulation.

This work was supported by grants from the American Cancer Society (CA-98-267) and the Department of Defense (CA-98-547) to R. B. Hazan. The government has certain rights in the invention.

### 1. INTRODUCTION

The present invention relates to methods for modulating the migratory, invasive and metastatic properties of cells expressing N-cadherin for the treatment of proliferative disorders including, but not limited to, cancers such as melanomas, breast, and prostate cancer. The invention further relates to drug screening assays designed to identify compounds that modulate N-cadherin activity and the use of such compounds in the treatment of disorders involving N-cadherin modulated cell migration, invasion, and metastasis. The invention also relates to methods for diagnosis and prognosis of disorders such as cancer that rely on detection of N-cadherin expression levels. The invention is based on the discovery that N-cadherin expression increases the migratory, invasive and metastatic properties of cells. It is additionally based on the discovery that increased N-cadherin expression sensitizes cells to growth factors such as FGF-2 and increases matrix metalloproteinase-9 (MMP-9) accumulation.

### 2. BACKGROUND OF INVENTION

The misregulation of cellular growth and proliferation is clearly a primary cause in the development of malignant tumors (Aaronson, S A, 1991, Science 254:1146-1153; Hanahan D, et al., 2000, Cell 100:57-70). Additional events must take place however, to endow cells with the ability to invade and metastasize (Liotta L A, et al., 1991, Cell 64:327-336; Woodhouse E C, et al., 1997, Cancer 80(8S):1529-1537). Metastasis is a complex process involving the detachment of cells from a primary tumor mass, crossing tissue boundaries such as basement membrane and interstitial stroma, entry and survival in the circulation, attachment to endothelium and penetration of intercellular junctions and subendothelial matrix to form new tumors at distant sites (Nicholson G, 1989, Curr Opin Cell Biol 1: 1009-1019; Blood C, et al., 1990, Biochem Biophys Acta 1032:89118).

It is apparent that these tasks involve the capacity of tumor cells to form dynamic adhesive interactions with different host cell surfaces. While this process is poorly understood, it is probable that new cell surface adhesion receptors become expressed which promote dynamic tumor cell interactions and facilitate their movement and that adhesion molecules that form stable epithelial-type adhesions become suppressed. It has also become apparent that in addition to this mechanical function, adhesion molecules fulfill much more complex functions which may result in the cell acquiring the ability to proliferate and invade host tissues.

The well characterized cadherins include P-, N-, and R-cadherin, which together are members of a closely related subfamily known as the “classic” cadherins. (Kemler R, 1992, Semin Cell Biol 3:149-155; Takeichi M, 1990, Ann Rev Biochem 59:237-252). The classic cadherins are differentially expressed during normal embryonic development, suggesting that they may have distinct functions. E- and P-cadherins are found primarily in epithelia where they promote the tight cell-cell associations known as adherins junctions (Takeichi M, 1991, Science 252:1451-1455). In contrast, N-cadherin is found primarily in neural tissues and fibroblasts (Hatta K et al., 1987, Dev. Biol 120:215-227; Hatta K et al., 1986, Nature 320:447-449; Redies C et al., 1993, Dev Dyn 197:26-39) where it is thought to mediate a less stable and more dynamic form of cell-cell adhesion (Bixby J L et al., 1990, J Cell Biol 110:1253-1260).

To infiltrate host tissues, cancer cells of epithelial origin have to separate from the tumor mass by breaking their cell-cell contacts, known as adherens junctions (Frixen et al., 1991, J. Cell Biol. 113:173-185; Vleminckx et al., 1991, Cell 66:107-119; Frixen and Nagamine, 1993, Cancer Res 53:3618-3623). Consistent with this hypothesis, the cell adhesion molecule E-cadherin, which is the adhesive component of adherens junctions, is notably absent or dysfunctional in most of the advanced, undifferentiated, and aggressive breast and other epithelial carcinomas (Perl et al., 1998, Nature 392:190-193; Christofori and Semb, 1999, Trends in Biochem 24:73-76). These and other findings support the model in which the loss of E-cadherin-based cell adhesion is considered to be an important factor in tumor invasiveness (Behrens et al., 1989, J. Cell. Biol.108:2435-2447; Frixen et al., 1991, J. Cell. Biol. 113:173-185; Vleminckx et al., 1991, Cell 66:107-119; Takeichi, 1993, Curr. Opin. Cell Biol. 5:806-811; Birchmeier and Behrens. 1994, Bio. Chim. Biophys. Acta 1198:11-26).

More recent evidence indicates that a gain of expression of another adhesion molecule, N-cadherin, in tumor cells is associated with an increased invasive potential. Previous studies have shown that N-cadherin is upregulated in more invasive and less differentiated breast cancer cell lines that lacked E-cadherin expression (Hazan et al., 1997, Cell Adhes. Commun.4:399-41 1). N-cadherin was also reported to induce a mesenchymal-scattered phenotype associated with reduced E- and P-cadherin levels in a squamous cell carcinoma cell line (Islam et al., 1996, J. Cell. Biol. 135:1643-1654). E-cadherin has been shown to promote only tight cell-cell adhesion, restricting cell movement, whereas N-cadherin has been postulated to promote both stable and labile cellular interactions that facilitate dynamic processes such as neurite outgrowth and cell migration (Hatta et al., 1987, Dev. biol.120:215-227; Bixby and Zhang, 1990, J. Cell. Biol. 110:1253-1260; Doherty et al.,1991, Mol. Cell Neurosci 8:99-111; Riehl et al., 1996, Neuron, 17:837-848).

Previous studies indicated that N-cadherin is upregulated in more invasive and less differentiated breast cancer cell lines that lacked E-cadherin expression (Hazan R B et al., 1997, Cell Adhes. Commun 4:399-411). N-cadherin was also reported to induce a mesenchymal-scattered phenotype associated with reduced E- and P-cadherin levels in a squamous cell carcinoma cell line (Islam S et al., 1996, J. Cell Biol 135:1643-1654). E-cadherin has been shown to promote only tight cell-cell adhesion, restricting cell movement, whereas N-cadherin has been postulated to promote both stable and labile cellular interactions that facilitate dynamic processes such as neurite outgrowth and cell migration (Hatta K et al., 1987, Dev. Biol 120:215-227; Bixby J L et al., 1990, J. Cell Biol 110: 1253-1260; Doherty P et al., 1991, Neuron 6:247-258; Riehl R et al., 1996, Neuron 17:837-848).

Kim J et al., using a variety of E- and N-cadherin chimeras in squamous epithelial cells, have recently reported that a 69-amino acid portion of the EC-4 region of N-cadherin is necessary and sufficient to promote both an epithelial to mesenchymal transition and increased cell motility (Kim J et al., 2000, J Cell Biol 151:1193-1205).

Vimentin, an intermediate filament protein expressed by mesenchymal cells, has been implicated in epithelial-to-mesenchymal transition. It has been proposed that the expression of vimentin and N-cadherins may be linked. A recent report indicates, however, that in squamous epithelial cells the expression of N-cadherin and vimentin do not effect the expression of the other (Islam S et al., 2000, J Cell Biochem 78:141-150).

The present invention relates to the discovery that the expression of N-cadherin in the weakly invasive and poorly metastatic (Kern F et al., 1994, Breast Cancer Res. Treat 31:153-165), E-cadherin-expressing breast cancer cell line, MCF-7, endows these cells with invasive and metastatic properties. The results demonstrate for the first time a direct correlation between expression N-cadherin and the metastatic potential of cancer cells. Moreover, N-cadherin expression sensitizes the cells to FGF-2-induced increases in cell migration, in vitro invasion, and MMP-9 production.

In order to provide cancer treatment and survival, methods for decreasing the metastatic potential of cancer cells are needed. The present invention fulfils these needs and further provides other related advantages.

### 3. SUMMARY OF THE INVENTION

The present invention relates to methods for modulating the migratory, invasive, or metastatic potential of cells expressing N-cadherin involving the administration of inhibitors of N-cadherin activity, including small molecules, large molecules, and antibodies. The invention also provides for compounds and nucleotide sequences that can be used to modulate N-cadherin gene expression.

The invention further provides diagnostic and prognostic assays that rely of the detection of N-cadherin expression levels. The invention is based on the observation that the level of N-cadherin expressed within the cell correlates with the metastatic potential of the cell. Thus, biological samples derived from a patient may be assayed for levels of N-cadherin expression, wherein increased levels of N-cadherin expression signify an increased metastatic potential. In addition, the assays of the invention may be used to monitor the progression of the disease within a patient.

The invention relates to assays designed to screen for compounds that modulate the biological activity of N-cadherin, i.e., compounds that act as agonists and antagonists of N-cadherin activity expressed by cells, such as cancer cells. The invention also relates to assays designed to screen for compounds that modulate N-cadherin gene expression. For example, cell-based assays can be used to screen for compounds that modulate N-cadherin transcription such as compounds that modulate expression, production or activity of transcription factors involved in N-cadherin gene expression; antisense and ribozyme polynucleotides that modulate translation of N-cadherin mRNA and polynucleotides that form triple helical structures with the N-cadherin regulatory region and inhibit transcription of the N-cadherin gene.

Identified compounds may be used in the treatment of disorders where the migratory, invasive, or metastatic activity of cells, contributes to the development of such disorders. Such disorders include, but are not limited to cancer of the breast or prostate and melanomas, for example, where inhibition of migratory, invasive, and/or metastatic activity using, for example, N-cadherin antagonists would be desired. In addition, agonist of N-cadherin activity may be used for enhancement of wound healing, promotion of neuronal cell growth and repair and treatment of developmental defects, for example, where induction of cell migration would be desired.

### 4. BRIEF DESCRIPTION OF THE FIGURES

FIG. 1. E- and N-cadherin expression and localization in N-cadherin MCF-7 transfectants and controls cells. (A) Triton X-100 cell lysates (20 μg of protein) from parental MCF-7 (lane 1), a vector-transfected clone (Neo-5; lane 2), N-cadherin mass cultures (N-cad-mass; lane 3), N-cadherin-transfected clones (N-cad-1 5, N-cad-8, N-cad-5, and N-cad-17; lanes 4-7, respectively), and MDA-MB-435 (lane 8) were immunoblotted with mAbs to either E-(top panel) or N-cadherin (bottom panel). (B) N-cad-5, N-cad-17, N-cad-mass cells, and parental MCF-7 cells were plated on collagen-coated cover slips, fixed and stained with monoclonal anti-E-(top) and anti-N-cadherin antibodies (bottom).

FIG. 2. N-cadherin promotes coaggregation of N-cadherin-transfected MCF-7 cells with N-cadherin-transfected L-cells. L-cells and L-E-cadherin and L-N-cadherin cells, labeled with the flourescent dye diO (green) were coaggregated with diI-labeled (red) MCF-7 cells (A, B, and C, respectively) or N-cad-5 cells (D, E, and F respectively). Yellow appears when green and red are superimposed, indicating coaggregation.

FIG. 3. N-cadherin promotes cell migration and invasion. (A) Cell motility through uncoated filters and (B) through Matrigel-coated filters was measured 18 h after plating. The migrating cells were stained (see Materials and Methods), visualized by microscopy, and triplicate filters were counted in three individual experiments. The numbers represent mean±SD for three experiments. (B) Cells that invaded the Matrigel layer were fixed, stained, and photographed; each panel represents an example of three to six replicates. Because of clustering of migrating cells on the underside of filter, no counting of single cells was possible.

FIG. 4. Cell migration is inhibited by a function-blocking N-cadherin antibody. (A) Cell motility through uncoated filters was measured 18 h after plating in the absence (top) or presence of 40 μg/ml purified anti-N-cadherin antibodies (middle) or 40 μg/ml control immunoglobulins (bottom). The migrating cells were stained (Materials and Methods), visualized by microscopy of triplicate filters, and (B) were counted in three individual experiments. The numbers represent mean±SD for three experiments.

FIG. 5. Histopathology of metastatic lesions in nude mice. 5-μm sections of the pancreas of mice injected with N-cad-17 cells were stained with either H&E (A) or cytokeratin antibodies, followed by DAB detection (B). Cytokeratin immunoreactivity of the N-cad-17 primary tumor (C) was compared with the pancreatic lesion produced by this tumor in B. Sections from the salivary gland of mice injected with N-cad-5 cells were stained with anticytokeratin antibodies, followed by DAB detection (D) and sections from the omentum (E) and primary tumor of N-cad-5-injected mice (F) were stained with FITC-conjugated cytokeratin antibodies. N-cad-8 metastatic lesions were detected in lung sections by H&E (G) and cytokeratin/DAB detection (H). N-cad-15 cells were found in the lumbar spinal muscle (I) using double flourescent staining with anti-actin, followed by secondary antibodies coupled to rhodamine (red) and FITC-conjugated anticytokeratin antibodies (green). Bar: (A) 100 μm; (G and H) 50 μm; (B, D, and E) 33 μm; (C, F, and I) 20 μm.

FIG. 6. The N-cadherin transgene is present in tumor cells microdissected from liver metastases. (A) Histology of N-cad-17 tumor infiltrates into the mouse liver stained by H&E shown at low (top panel) and high (bottom panel) magnification. (B) The DNA of cells dissected from MCF-7, N-cad-5 and N-cad-17 primary tumors (lanes 1, 3, and 4, respectively) or from liver sections (lanes 2, 5, and 6, respectively) was subjected to PCR and Southern blotting using primers designed to amplify a 200-bp fragment from the transfected human N-cadherin cDNA (see Materials and Methods). Human N-cadherin plasmid DNA was used as a positive control (lane 7). Bar: (A) 100 μm; (B) 33 μm.

FIG. 7. E- and N-cadherin expression in metastatic lesions. Sections of salivary glands (Sal, top left), pancreas (top right); axillary lymph nodes (LN, bottom right), from mice injected with N-cad-5 and N-cad-17 (Table I) were stained with—and E-cadherin antibodies using a secondary DAB detection. The metastatic cells (mets) express high levels of both N-and E-cadherin. Staining of primary tumors from MCF-7-injected mice (bottom left) shows a positive reaction with E-cadherin but not with N-cadherin antibodies. Bar, 20 μm.

FIG. 8. Stimulates cell migration of N-cadherin-expressing cells. The motility of control (Neo-mass) and N-cadherin-expressing cells (N-cad-mass, N-cad-5, and N-cad-17), pretreated for 24 h with 10 ng/ml of FGF-2 or left untreated, was measured 5 h after inoculation into the Transwell chambers. (A) The migrating cells were fixed, stained, and photographed; each panel represents an example of 3-6 replicates. (B) Bar graphs represent quantitation of triplicate wells. The experiment was repeated six times.

FIG. 9. FGF-2 stimulates Matrigel invasion of N-cadherin-expressing cells and expression of MMP-9. (A) Invasion through Matrigel-coated filters of control and N-cadherin-expressing cells, pretreated for 24 h with 10 ng/ml of FGF-2 or left untreated, as measured 5 h after inoculation into Transwell chambers. The invading cells were stained (see Materials and Methods) and photographed using a digital microscope camera. Each panel illustrates a sample representing three to six filters. (B) Gelatinolytic activity of conditioned media of control (Neo-mass) (lanes 1 and 2) or N-cadherin-transfected MCF-7 cells (N-cad-mas, N-cad-5, and N-cad-17; lanes 3-8) that were either treated with FGF-2, or left untreated, as indicated. (C) Gelatinolytic activity of conditioned media of Neo-mass, N-cad-mass, N-cad-5, and N-cad- 17 that were pretreated for 24 h either with 10 ng/ml of FGF-2 (lanes 1, 3, 5 and 7, respectively) or 5 μg/ml insulin (lanes 2, 4, 6, and 8, respectively).

FIG. 10. N-cadherin promotes adhesion of MCF-7 cells to HUVEC cells. (A) Each panel shows an unstained (and therefore not visible) monolayer of HUVEC cells incubated with control Neo-5 (A), N-cad-5 (B), or N-cad-17 (C) cells labeled with flourescent dye (Fast diO) (See Materials and Methods). D represents N-cadherin immunoblotting of cell extracts from HUVEC (right) compared with Neo-5 (left) and N-cad-5 (middle) control cells.

FIG. 11. N-cadherin associates with the FGF receptor through its extracellular domain. 293T cells (N-cadherin positive) transfected with either FGFR (lanes 2 and 3) or with vector alone (lane 1) were treated with or without FGF-2, immunoprecipitated with anti-N-cadherin antibodies, and probed with an anti-FGFR antibody.

FIG. 12. N-cadherin synergizes with FGF-2 in promoting MAPK activation and MMP-9 production. A. MCF-7 breast cancer cells expressing either control vector (control) or N-cadherin (N-cad-5) were treated with or without FGF-2 and the relative activation of MAPK was assessed by blotting the lysates with anti phospho-MAPK (P-MAPK) (upper panel A, lanes 1-4) against the total levels of MAPK in the same lysates (T-MAPK) (lower panel A, lanes 1-4). B. MCF-7 breast cancer cells expressing N-cadherin (N-cad-5) were treated with or without FGF-2 in the absence or presence of a potent inhibitor of MAPK (PD 90859) and cell lysates were blotted with anti-P-MAPK antibodies (lower panel B) or assayed for MMP (Matrix Metallo Protease) production (upper panel B).

FIG. 13. MMP-9 induction by FGF-2 in NIH-3T3 cells. (A) Serum-starved control NIH-3T3 (lanes 1, 2) and FGFR-1 transfected NIH-3T3 (lanes 3, 4) were untreated (lanes 1, 3) or treated (lanes 2, 4) with 10 ng/mL FGF-2 for 24 hours and conditioned media from these cells was analyzed for zymmographic activity.

FIG. 14. Tissue sections derived from normal, benign (tumor-1) and advanced prostate cancer (tumor-2) were stained with anti-cadherin (left panel) or N-cadherin (right panel) and followed by DAB color reaction (brown). Note up regulation of N-cadherin in most advanced tumor (tumor-2).

FIG. 15. E- and N-cadherin reactivity with paraffin-embedded sections of an invasive breast carcinoma is shown in panels A and B at low and high magnifications respectively. (A) E-cadherin stains the well-differentiated part of the tumor while (B) N-cadherins stains the de-differentiated breast tumor cells well within the same tumor.

### 5. DETAILED DESCRIPTION OF THE INVENTION

The present invention relates to methods for modulating the migratory, invasive, and metastatic properties of cells involving the regulation of N-cadherin activity. The invention is based on the discovery that N-cadherin expression increases the migratory, invasive and metastatic properties of cells. It is additionally based on the discovery that N-cadherin expression sensitize cells to growth factors (e.g. FGF-2) and increases metalloproteinase (e.g. MMP-9) accumulation.

The present invention encompasses screening assays designed for the identification of modulators, such antagonists of N-cadherin activity. The invention further relates to the use of such modulators in the treatment of disorders based on N-cadherin mediated migratory, invasive, and/or metastatic activity. Such disorders include, but are not limited to, cancer.

Various aspects of the invention may be better understood in view of the following detailed descriptions and examples.

### 5.1. Screening Assays for Compounds Useful in Modulating the Activity of N-Cadherin

The present invention relates to screening assay systems designed to identify compounds or compositions that modulate N-cadherin activity and/or N-cadherin gene expression, and thus, may be useful for modulation of cell migration, invasion, and metastasis.

### 5.1.1. Recombinant Expression of N-Cadherin

For purposes of developing screening assays designed to identify compounds or compositions that modulate N-cadherin activity it may be necessary to recombinantly express the N-cadherin protein. The cDNA sequence and deduced amino acid sequence of N-cadherin has been characterized from several species including humans. For recombinant expression of N-cadherin, sequences derived from any species may be used. The human N-cadherin is available from GenBank/EMBL/DDBJ under accession number S42303).

N-cadherin nucleotide sequences may be isolated using a variety of different methods known to those skilled in the art. For example, a cDNA library constructed using RNA from a tissue known to express N-cadherin can be screened using a labeled N-cadherin probe. Alternatively, a genomic library may be screened to derive nucleic acid molecules encoding the N-cadherin protein. Further, N-cadherin nucleic acid sequences may be derived by performing a polymerase chain reaction (PCR) using two oligonucleotide primers designed on the basis of known N-cadherin nucleotide sequences. The template for the reaction may be cDNA obtained by reverse transcription of mRNA prepared from cell lines or tissue known to express N-cadherin.

N-cadherin protein, polypeptides and peptide fragments, mutated, truncated or deleted forms of N-cadherin and/or N-cadherin fusion proteins can be prepared for a variety of uses, including but not limited to the generation of antibodies, the identification of other cellular gene products involved in the regulation of N-cadherin mediated activities such as cell migration invasion and metastases, and the screening for compounds that can be used to modulate such activities. N-cadherin fusion proteins include fusions to an enzyme, fluorescent protein, a polypeptide tag or luminescent protein which provide a marker function.

While the N-cadherin polypeptides and peptides can be chemically synthesized (e.g., see Creighton, 1983, Proteins: Structures and Molecular Principles, W.H. Freeman & Co., N.Y.), large polypeptides derived from N-cadherin and the full length N-cadherin itself may be advantageously produced by recombinant DNA technology using techniques well known in the art for expressing a nucleic acid containing N-cadherin gene sequences and/or coding sequences. Such methods can be used to construct expression vectors containing the N-cadherin nucleotide sequences and appropriate transcriptional and translational control signals. These methods include, for example, in vitro recombinant DNA techniques, synthetic techniques, and in vivo genetic recombination. (See, for example, the techniques described in Sambrook et al., 1989, Molecular Cloning. A Laboratory Manual, Second Edition, Cold Spring Harbor Press, N.Y.; and Ausubel et al., 1989, Current Protocols in Molecular Biology, Green Publishing Associates and Wiley Interscience, N.Y.).

A variety of host-expression vector systems may be utilized to express the N-cadherin nucleotide sequences. Where the N-cadherin peptide or polypeptide is expressed as a soluble derivative (e.g., peptides corresponding to the extracellular, transmembrane or cytoplasmic domain) and is not secreted, the peptide or polypeptide can be recovered from the host cell. Alternatively, where the N-cadherin peptide or polypeptide is secreted the peptide or polypeptides may be recovered from the culture media. However, the expression systems also include engineered host cells that express N-cadherin or functional equivalents, anchored in the cell membrane. Purification or enrichment of the N-cadherin from such expression systems can be accomplished using appropriate detergents and lipid micelles and methods well known to those skilled in the art. Such engineered host cells themselves may be used in situations where it is important not only to retain the structural and functional characteristics of the N-cadherin, but to assess biological activity, i.e., in drug screening assays.

The expression systems that may be used for purposes of the invention include but are not limited to microorganisms such as bacteria transformed with recombinant bacteriophage, plasmid or cosmid DNA expression vectors containing N-cadherin nucleotide sequences; yeast transformed with recombinant yeast expression vectors containing N-cadherin nucleotide sequences or mammalian cell systems or insect cell systems harboring recombinant expression constructs containing promoters derived from the genome of mammalian or insect cells or from mammalian or insect viruses.

Appropriate expression systems can be chosen to ensure that the correct modification, processing and sub-cellular localization of the N-cadherin protein occurs. To this end, eukaryotic host cells which possess the ability to properly modify and process the N-cadherin protein are preferred. For long-term, high yield production of recombinant N-cadherin protein, such as that desired for development of cell lines for screening purposes, stable expression is preferred. Rather than using expression vectors which contain origins of replication, host cells can be transformed with DNA controlled by appropriate expression control elements and a selectable marker gene, i.e., tk, hgprt, dhfr, neo, and hygro gene, to name a few. Following the introduction of the foreign DNA, engineered cells may be allowed to grow for 1-2 days in enriched media, and then switched to a selective media. Such engineered cell lines may be particularly useful in screening and evaluation of compounds that modulate the endogenous activity of the N-cadherin gene product.

### 5.1.2. Non-Cell Based Assays

In accordance with the invention, non-cell based assay systems may be used to identify compounds that interact with, i. e., bind to N-cadherin, and regulate the activity of N-cadherin. Such compounds may act as inhibitors of N-cadherin activity and may be used to inhibit migration, invasion and/or metastasis of cancer cells. Recombinant N-cadherin, including peptides corresponding to different functional domains, or N-cadherin fusion proteins, may be expressed and used in assays to identify compounds that interact with N-cadherin.

To this end, soluble N-cadherin may be recombinantly expressed and utilized in non-cell based assays to identify compounds that bind to N-cadherin. Recombinantly expressed N-cadherin polypeptides or fusion proteins containing one or more of the N-cadherin functional domains may be prepared as described above, and used in the non-cell based screening assays. For example, the full length N-cadherin, or a soluble truncated N-cadherin, e.g., in which the one or more of the cytoplasmic and transmembrane domains is deleted from the molecule, a peptide corresponding to the extracellular domain, or a fusion protein containing the N-cadherin extracellular domain fused to a protein or polypeptide that affords advantages in the assay system (e.g., labeling, isolation of the resulting complex, etc.) can be utilized. Where compounds that interact with the cytoplasmic domain are sought to be identified, peptides corresponding to the N-cadherin cytoplasmic domain and fusion proteins containing the N-cadherin cytoplasmic domain can be used.

The N-cadherin protein may also be one which has been fully or partially isolated from cell membranes, or which may be present as part of a crude or semi-purified extract. As a non-limiting example, the N-cadherin protein may be present in a preparation of cell membranes. In particular embodiments of the invention, such cell membranes may be prepared using methods known to those of skill in the art.

The principle of the assays used to identify compounds that bind to N-cadherin involves preparing a reaction mixture of the N-cadherin and the test compound under conditions and for time sufficient to allow the two components to interact and bind, thus forming a complex which can be removed and/or detected in the reaction mixture. The identity of the bound test compound is then determined.

The screening assays are accomplished by any of a variety of commonly known methods. For example, one method to conduct such an assay involves anchoring the N-cadherin protein, polypeptide, peptide, fusion protein or the test substance onto a solid phase and detecting N-cadherin/test compound complexes anchored on the solid phase at the end of the reaction. In one embodiment of such a method, the N-cadherin reactant is anchored onto a solid surface, and the test compound, which is not anchored, may be labeled, either directly or indirectly.

In practice, microtitre plates conveniently can be utilized as the solid phase. The anchored component is immobilized by non-covalent or covalent attachments. The surfaces may be prepared in advance and stored. In order to conduct the assay, the non-immobilized component is added to the coated surfaces containing the anchored component. After the reaction is completed, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the solid surface; e.g., using a labeled antibody specific for the previously non-immobilized component.

Alternatively, a reaction is conducted in a liquid phase, the reaction products separated from unreacted components using an immobilized antibody specific for N-cadherin protein, fusion protein or the test compound, and complexes detected using a labeled antibody specific for the other component of the possible complex to detect anchored complexes.

In a further embodiment of the invention, non-cell based assay systems may be used to identify compounds that inhibit the interaction between the FGFR and N-cadherin, thereby regulating N-cadherin mediated signal transduction. Such compounds may act as inhibitors of N-cadherin activity and may be used to inhibit migration, invasion and/or metastasis of cancer cells. Full length N-cadherin and FGFR proteins can be utilized to screen for compounds that inhibit the protein interaction observed between the two proteins. Alternatively, the N-cadherin and FGFR may be fully or partially isolated from cell membranes, or which may be present as part of a crude or semi-purified extract. Alternatively, recombinant N-cadherin, including peptides corresponding to the extracellular domain of N-cadherin, and recombinant FGFR, including peptides corresponding to those domains known to be required for N-cadherin binding may be expressed and used in assays to identify compounds that interact with N-cadherin.

The principle of the assays used to identify compounds that inhibit binding of N-cadherin /FGFR complex formation involves preparing a reaction mixture of the N-cadherin, FGFR and the test compound under conditions and for a time sufficient to allow the components to interact and bind, thus forming a complex which can be removed and/or detected in the reaction mixture. The ability of the test compound to inhibit N-cadherin and FGFR binding is then determined by detecting a decrease in the level of N-cadherin/FGFR complexes in the presence of the test compound as compared to control.

Such screening assays are accomplished by any of a variety of commonly known methods. For example, one method to conduct such an assay involves anchoring either N-cadherin or FGFR onto a solid phase and detecting N-cadherin/FGFR complexes anchored on the solid phase at the end of the reaction. In one embodiment of such a method, either the N-cadherin or FGFR reactant is anchored onto a solid surface, while the non-immobilized reactant is labeled, either directly or indirectly. The detection of N-cadherin/FGFR complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the solid surface; e.g., using a labeled antibody specific for the previously non-immobilized component.

Alternatively, a reaction is conducted in a liquid phase, the reaction products are separated from unreacted components using an immobilized antibody specific for either N-cadherin or FGFR protein, and N-cadherin/FGFR complexes are detected using a labeled antibody specific for the other component of the possible complex to detect anchored complexes.

In another embodiment of the invention, computer modeling and searching technologies will permit identification of potential modulators of N-cadherin activity. As described in detail below, N-cadherin is demonstrated to interact directly with the fibroblast growth factor receptor (FGFR) and this interaction potentiates N-cadherin mediated cell migration and invasion. Thus, for example, based on the knowledge of the N-cadherin sites involved in complexes between N-cadherin and FGFR potential modulators of N-cadherin activity may be identified.

The three dimensional geometric structure of the active binding site may be determined using known methods, including x-ray crystallography, which can determine a complete molecular structure. On the other hand, solid or liquid phase NMR can be used to determine certain intramolecular distances. Any other experimental method of structure determination can be used to obtain the partial or complete geometric structure of the N-cadherin/FGFR binding site. The crystal structure of N-cadherin has been reported by Shapiro et al., (1995, Nature 374:327-37) and Tamura et al., (1998, Neuron, 20:1153-1163). In addition, the crystal structure of FGFR is known (Schellessinger et al., 2000, Molecular Cell. 6:743-750).

Having determined the structure of the N-cadherin/FGFR binding site, candidate modulating compounds can be identified by searching databases containing compounds along with information on their molecular structure. Such a search seeks compounds having structures that match the binding site structure and that interact with the groups defining the binding site. Such a search can be manual, but is preferably computer assisted. These compounds found from this search are potential N-cadherin modulating compounds.

Alternatively, these methods can be used to identify improved modulating compounds from an already known modulating compounds such as N-cadherin antibodies. The composition of the known compound can be modified and the structural effects of modification can be determined using experimental and computer modeling methods applied to the new composition. The altered structure is then compared to the active site structure of the compound to determine if an improved fit or interaction results. In this manner systematic variations in composition, such as by varying side groups, can be quickly evaluated to obtain modified modulating compounds or substrates of improved specificity or activity.

### 5.1.3.Cell Based Assays

In accordance with the invention, a cell based assay system can be used to screen for compounds that modulate the activity of N-cadherin. In accordance with the invention, a cell-based assay system can be used to screen for compounds that modulate the activity of N-cadherin and thereby, modulate the migratory, invasive, and/or metastatic properties of cells. Also in accordance with the invention, a cell-based assay system can be used to screen for compounds that modulate the interaction of N-cadherin with FGFR and thereby, modulate the migratory, invasive, and/or metastatic properties of cells.

Cells that endogenously express N-cadherin can be used to screen for compounds. Such cells include, for example, MDAB435, MDAB436 and BT549. Alternatively, cell lines, such as 293T cells, NIH-3T3, MCF-7, and the like, genetically engineered to express N-cadherin can be used for screening purposes. Preferably, host cells genetically engineered to express a functional N-cadherin are those capable of signal transduction in response to contact with growth factors such as FGF-2. Further, ooyctes or liposomes engineered to express the N-cadherin protein may be used in assays developed to identify modulators of N-cadherin activity.

According to the invention, “N-cadherin activity” refers to N-cadherin mediated cell aggregation, adhesion, migration, invasion and/or metastasis. Such activities may be measured using a variety of different assay methods designed to measure, for example, cell migration, aggregation, adhesion and invasion. “N-cadherin activity” also refers to N-cadherin/FGF-2 mediated signal transduction that can be assayed by measuring the activity and/or expression of components in the FGF-2 signal transduction pathway. Such assays include detection of N-cadherin/FGFR complexes, increased MMP-9 expression and/or activation of MAPK activity.

The present invention provides for methods for identifying a compound that inhibits N-cadherin activity comprising (i) contacting a cell expressing N-cadherin with a test compound and measuring the level of N-cadherin activity; (ii) in a separate experiment, contacting a cell expressing N-cadherin with the test compound vehicle control and measuring the level of N-cadherin activity, where the conditions are essentially the same as in part (i) and then (iii) comparing the level of N-cadherin activity measured in part (i) with the level of N-cadherin activity in part (ii), wherein a decrease level of N-cadherin activity in the presence of the test compound indicates that the test compound is a N-cadherin inhibitor.

The present invention provides for methods for identifying a compound that activates N-cadherin activity comprising (i) contacting a cell expressing N-cadherin with a test compound and measuring the level of N-cadherin activity; (ii) in a separate experiment, contacting a cell expressing N-cadherin with the test compound vehicle control and measuring the level of N-cadherin activity, where the conditions are essentially the same as in part (i) and then (iii) comparing the level of N-cadherin activity measured in part (i) with the level of N-cadherin activity in part (ii), wherein an increase in the level of N-cadherin activity in the presence of the test compound indicates that the test compound is a N-cadherin activator.

The present invention also provides for methods for identifying a compound that inhibits N-cadherin activity comprising (i) contacting a cell expressing N-cadherin with a test compound in the presence of FGF-2 and measuring the activity of N-cadherin; (ii) in a separate experiment, contacting a cell expressing N-cadherin with the test compound vehicle control in the presence of FGF-2 and measuring the activity of N-cadherin, where the conditions are essentially the same as in part (i) and then (iii) comparing the level of activity measured in part (i) with the level of activity in part (ii), wherein a decrease level of activity in the presence of the test compound indicates that the test compound is an inhibitor of N-cadherin activity.

The present invention also provides for methods for identifying a compound that activates N-cadherin activity comprising (i) contacting a cell expressing N-cadherin with a test compound in the presence of FGF-2 and measuring the activity of N-cadherin; (ii) in a separate experiment, contacting a cell expressing N-cadherin with the test compound vehicle control in the presence of FGF-2 and measuring the activity of N-cadherin, where the conditions are essentially the same as in part (i) and then (iii) comparing the level of activity measured in part (i) with the level of activity in part (ii), wherein an increase in the level of activity in the presence of the test compound indicates that the test compound is an activator of N-cadherin activity.

In utilizing such cell systems, the cells expressing the N-cadherin protein are exposed to a test compound or to vehicle controls (e.g., placebos). After exposure, the cells can be assayed to measure the activity of N-cadherin. Those skilled in the art will be able to determine operative and optimal assay conditions by employing routine experimentation.

The ability of a test compound to modulate N-cadherin activity may be assayed using a variety of different methods. For example, coaggregation assays may be used to measure the ability of a test compound to modulate cellular aggregation. In such assays, single cell suspensions of cells expressing N-cadherin are mixed in the presence and absence of the test compound. The mixed cells are then visualized to determine whether cell aggregation has occurred. In a specific embodiment of the invention, the cells may be labeled with a fluorescent dye prior to mixing, to facilitate visualization of aggregating cells. The level of cell aggregation is measured in the presence of a test compound and compared to the level of cell aggregation observed in the absence of a test compound wherein a decrease in the level of cell aggregation in the presence of a test compound indicates identification of an inhibitor of N-cadherin activity, and wherein an increase in the level of cell aggregation in the presence of a test compound indicates identification of an activator of N-cadherin activity.

Alternatively, the ability of a test compound to modulate N-cadherin mediated adhesion of cells to endothelium may be measured. For example, human endothelium monolayers may be formed by plating HUVEC cells on gelatin coated cover slips. Cells expressing N-cadherin are then added to the endothelium monolayers and incubated for a time sufficient to allow adhesion to the monolayer. The level of cell adhesion is measured in the presence of a test compound and compared to the level of adhesion observed in the absence of a test compound wherein a decrease in the level of adhesion in the presence of a test compound indicates identification of an inhibitor of N-cadherin activity and wherein an increase in the level of adhesion in the presence of a test compound indicates identification of an activator of N-cadherin activity.

In yet another embodiment of the invention, cell migration and invasion through Matrigel-coated filters as described in Albini et al., (Albini A et al., 1987, Cancer Res 47:3239-3245) can be measured to identify modulators of N-cadherin activity. The level of cell migration and invasion through Matrigel-coated filters is measured in the presence of a test compound and compared to the level of cell migration and invasion observed in the absence of a test compound wherein a decrease in the level of cell migration and invasion in the presence of a test compound indicates identification of an inhibitor of N-cadherin activity and wherein an increase in the level of cell migration and invasion in the presence of a test compound indicates identification of an activator of N-cadherin activity .

The ability of a test molecule to modulate the activity of N-cadherin in the presence of FGF-2 may be measured using standard biochemical and physiological techniques. Responses such as activation or suppression of matrix metalloproteinase-9 activity or MAPK activity can be measured. Levels of matrix metalloproteinase-9 can be measured using, for example, substrate gel electrophoresis (Zymography) as described in Nakajima et al. Nakajima I et al., 1995, Br. J. Cancer. 71:1039-1045). Levels of MAPK activity can be measured as described in the Example 6, infra.

Further, assays designed to measure the level of N-cadherin/FGFR complexes within a cell may be used to identify modulators of N-cadherin activity. The detection of N-cadherin/FGFR complexes may be achieved by any of a variety of techniques well known in the art. Specifically, immunoassays such as immunoprecipitations may be used for detection of N-cadherin/FGFR complexes. The level of N-cadherin/FGFR complexes is measured in the presence of a test compound and compared to the level of N-cadherin/FGFR complexes observed in the absence of a test compound wherein a decrease in the level of N-cadherin/FGFR complexes in the presence of a test compound indicates identification of an inhibitor of N-cadherin activity and wherein an increase in the level of N-cadherin/FGFR complexes in the presence of a test compound indicates identification of an activator of N-cadherin activity.

### 5.1.4. Assay for Compounds that Regulate the Expression of N-Cadherin

In accordance with the invention, a cell based assay system can be used to screen for compounds that modulate the expression of N-cadherin within a cell. Assays may be designed to screen for compounds that regulate N-cadherin expression at either the transcriptional or translational level. In one embodiment, DNA encoding a reporter molecule can be linked to a regulatory element of the N-cadherin gene (Li et al., 19979, Gene 19:7-13) and used in appropriate intact cells, cell extracts or lysates to identify compounds that modulate N-cadherin gene expression. Reporter genes may include but are not limited to chloramphenicol acetyltransferase (CAT), luciferase, β-glucuronidase (GUS), growth hormone, or placental alkaline phosphatase (SEAP). Such constructs are introduced into cells thereby providing a recombinant cell useful for screening assays designed to identify modulators of N-cadherin gene expression.

Following exposure of the cells to the test compound, the level of reporter gene expression may be quantitated to determine the test compound's ability to regulate N-cadherin expression. Alkaline phosphatase assays are particularly useful in the practice of the invention as the enzyme is secreted from the cell. Therefore, tissue culture supernatant may be assayed for secreted alkaline phosphatase. In addition, alkaline phosphatase activity may be measured by calorimetric, bioluminescent or chemiluminescent assays such as those described in Bronstein, I. et al. (1994, Biotechniques 17:172-177). Such assays provide a simple, sensitive easily automatable detection system for pharmaceutical screening.

To identify compounds that regulate N-cadherin translation, cells or in vitro cell lysates containing N-cadherin transcripts may be tested for modulation of N-cadherin mRNA translation. To assay for inhibitors of N-cadherin translation, test compounds are assayed for their ability to modulate the translation of N-cadherin mRNA in in vitro translation extracts.

In an embodiment of the invention, the level of N-cadherin expression can be modulated using antisense or ribozyme approaches to inhibit or prevent translation of N-cadherin mRNA transcripts or triple helix approaches to inhibit transcription of the N-cadherin gene. Such approaches may be utilized to treat disorders such as cancer where inhibition of N-cadherin expression is designed to prevent cancer cell migration, invasion and metastasis.

Antisense approaches involve the design of oligonucleotides (either DNA or RNA) that are complementary to N-cadherin mRNA. The antisense oligonucleotides will bind to the complementary mRNA transcripts and prevent translation. Absolute complementarity, although preferred, is not required. One skilled in the art can ascertain a tolerable degree of mismatch by use of standard procedures to determine the melting point of the hybridized complex.

In yet another embodiment of the invention, ribozyme molecules designed to catalytically cleave N-cadherin mRNA transcripts can also be used to prevent translation of N-cadherin mRNA and expression of N-cadherin. (See, e.g., PCT International Publication W)90/11364, published Oct. 4, 1990; Sarver et al., 1990, Science 247:1222-1225). Alternatively, endogenous N-cadherin gene expression can be reduced by targeting deoxyribonucleotide sequences complementary to the regulatory region of the N-cadherin gene (i.e., the N-cadherin promoter and or enhancers) to form triple helical structures that prevent transcription of the N-cadherin gene in targeted cancer cells in the body. (See generally, Helene C. et al., 1991, Anticancer Drug Des. 6:569-584 and Maher L J, 1992, Bioassays 14:807-815).

The oligonucleotides of the invention, i.e., antisense, ribozyme and triple helix forming oligonucleotides, may be synthesized by standard methods known in the art, e.g., by use of an automated DNA synthesizer (such as are commercially available from Biosearch, Applied Biosystems, etc.). Alternatively, recombinant expression vectors may be constructed to direct the expression of the oligonucleotides of the invention. Such vectors can be constructed by recombinant DNA technology methods standard in the art. In a specific embodiment, vectors such as viral vectors may be designed for gene therapy applications where the goal is in vivo expression of inhibitory oligonucleotides in targeted cells.

### 5.1.5. Compounds that can be Screened in Accordance with the Invention

The assays described above can identify compounds which modulate N-cadherin activity. For example, compounds that affect N-cadherin activity include but are not limited to compounds that bind to N-cadherin, and either activate activities (agonists) or block activities (antagonists). Alternatively, compounds may be identified that do not bind directly to N-cadherin but are capable of altering N-cadherin activity by altering the activity of a protein that interacts with N-cadherin, such as FGFR, or a protein that is involved in N-cadherin/FGF-2 mediated signal transduction. Further, compounds that affect N-cadherin gene activity (by affecting N-cadherin gene expression, including molecules, e.g., proteins or small organic molecules, that affect transcription or interfere with splicing events so that expression of the full length or the truncated form of the N-cadherin can be modulated) can be identified using the screens of the invention.

The compounds which may be screened in accordance with the invention include, but are not limited to, small organic or inorganic compounds, peptides, antibodies and fragments thereof, and other organic compounds (e.g., peptidomimetics) that bind to N-cadherin and either increase the activity of N-cadherin (i.e., agonists) or inhibit the activity triggered by any of the known or unknown substrates of N-cadherin (i.e., antagonists). Compounds that bind to N-cadherin and either enhance N-cadherin activities (i.e., cell adhesion, co-aggregation, migration, invasion, metastases, activation of MMP-9 expression or MAPK activity), i.e., agonists, or compounds that inhibit N-cadherin activities, i.e., antagonists, will be identified. Compounds that directly activate or inhibit the cADPR Ca2+ signal transduction pathway in cells can be identified.

Compounds may include, but are not limited to, peptides such as, for example, soluble peptides, including but not limited to members of random peptide libraries (see, e.g., Lam K S et al., 1991, Nature 354:82-84; Houghten R et al., 1991, Nature 354:84-86); and combinatorial chemistry-derived molecular library made of D- and/or L- configuration amino acids, phosphopeptides (including, but not limited to, members of random or partially degenerate, directed phosphopeptide libraries; (see, e.g., Songyang Z et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope binding fragments thereof), and small organic or inorganic molecules.

Other compounds which may be screened in accordance with the invention include but are not limited to small organic molecules that affect the expression of the N-cadherin gene or some other gene involved in the N-cadherin signal transduction pathway, such as MMP-9 (e.g., by interacting with the regulatory region or transcription factors involved in gene expression); or such compounds that affect the activities of the N-cadherin or the activity of some other factor involved in modulating N-cadherin activity, such as for example, a protein that post-translationally modifies N-cadherin and thereby inactivates N-cadherin activities.

### 5.3. Diagnostic Prognostic Assays

In accordance with the invention, measurement of N-cadherin levels, i.e, protein or RNA levels, can be used for the diagnosis of diseases such as cancer. Moreover, the monitoring of N-cadherin levels can be used prognostically to stage the progression of the disease or the efficacy of drug treatment. The detection of N-cadherin levels in a sample from a patient can be accomplished by any of a number of methods. Such methods include immunoassays which include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few.

Such an immunoassay is carried out by a method comprising contacting a sample derived from a subject with an antibody immunoreactive with N-cadherin under conditions such that specific antigen-antibody binding can occur, and detecting or measuring the amount of any immunospecific binding to N-cadherin. In a specific aspect, such binding of antibody to samples, for example, can be used to detect the presence of N-cadherin wherein the detection of N-cadherin is an indication of a diseased condition. i.e., the presence of cancer cells with an increased metastatic potential. The levels of N-cadherin in a sample are compared to the levels present in an analogous sample from a subject not having the disorder.

Antibodies can be used in assays, such as the immunoassays listed above, to detect, prognose, diagnose, or monitor cancer in an individual, or monitor the treatment thereof. In particular, such an immunoassay is carried out by a method comprising contacting a sample derived from a subject with an antibody under conditions such that immunospecific binding can occur, and detecting or measuring the amount of any immunospecific binding by the antibody. In addition, reagents other than antibodies, such as, for example, nucleic acid molecules, polypeptides or chemical compounds that specifically bind to N-cadherin, can be used in assays to detect the expression of N-cadherin.

In a specific aspect, such binding of antibody to biological samples, can be used to detect expression of the protein wherein the expression of the protein is an indication of a diseased condition. The levels of expressed proteins are compared to levels relative to that present in an analogous sample from a portion of the body or from a subject not having the disorder.

The present invention also provides diagnostic and prognostic methods involving the detection of MMP-9 expression levels. As described herein, increased levels of MMP-9 correlates with an increased metastatic potential. In accordance with the invention, measurement of MMP-9 levels can be used for the diagnosis of diseases such as cancer. Moreover, the monitoring of MMP-9 levels can be used prognostically to stage the progression of the disease. The detection of MMP-9 levels in a sample from a patient can be accomplished by any of a number of methods, such as those described for N-cadherin, supra. In addition, assays designed to measure MMP-9 enzyme activity may be used to diagnose or prognose the metastatic potential of cells. Such assays include, zymography assays, such as those described in the working examples, which may be used to measure levels of secreted MMP-9 enzyme activity.

### 5.2. Compositions Containing Modulators of N-Cadherin and Their Uses

The present invention provides for methods of modulating cell migration, invasion and metastatic potential comprising contacting a cell with an effective amount of a N-cadherin cadherin modulating compound, such as a N-cadherin agonist or antagonist identified using the assays as set forth in Section 5.1 supra. An “effective amount” of the N-cadherin inhibitor, i.e., antagonist, is an amount that decreases cell migration, invasion and/or metastasis and/or that is associated with a detectable decrease in N-cadherin activity as measured by one of the above assays. An “effective amount” of the N-cadherin activator, i. e., agonist, is an amount that subjectively increases induced cell migration and/or that is associated with a detectable increase in N-cadherin activity as measured by one of the above assays.

The present invention further provides methods of modulating cell migration in a subject, comprising administering to the subject, a composition comprising a compound that modulates N-cadherin activity identified as set forth in Section 5.1 supra. The composition may comprise an amount of N-cadherin activator or inhibitor or modulators of N-cadherin expression. Accordingly, the present invention provides for compositions comprising N-cadherin activators and inhibitors.

The present invention provides for compositions comprising an effective amount of a compound capable of modulating the activity of N-cadherin, the expression of N-cadherin thereby regulating the migratory, invasive, and/or metastatic activity of cells, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical Sciences” by E. W. Martin.

The invention provides for treatment or prevention of various diseases and disorders associated with cell migration by administration of a compound that regulates the expression or activity of N-cadherin. Such compounds include but are not limited to N-cadherin antibodies; N-cadherin antisense nucleic acids, N-cadherin agonists and antagonists. In a non-limiting embodiment of the invention, disorders associated with increased cell migration, invasion and/or metastatic potential are treated or prevented by administration of a compound that inhibits N-cadherin activity. Such disorders include but are not limited to cancers, including but not limited to breast, prostate and melanomas. In addition, agonists of N-cadherin could be used for promotion of wound healing, nerve regeneration and birth associated developmental defects.

The compounds of the invention are preferably tested in vitro, and then in vivo for a desired therapeutic or prophylactic activity, prior to use in humans. For example, in vitro assays which can be used to determine whether administration of a specific therapeutic is indicated, include in vitro cell culture assays in which cells expressing N-cadherin are exposed to or otherwise administered a therapeutic compound and the effect of such a therapeutic upon N-cadherin activity is observed. In a specific embodiment of the invention the ability of a compound to regulate, i.e., activate or inhibit cell migration, invasion or metastatic potential may be assayed.

The invention provides methods of treatment and/or prophylaxis by administration to a subject of an effective amount of a compound of the invention. In a preferred aspect, the compound is substantially purified. The subject is preferably an animal, and is preferably a mammal, and most preferably human.

Various delivery systems are known and can be used to administer a compound capable of regulating N-cadherin activity, or N-cadherin expression, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432). Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Pulmonary administration can also be employed, e.g., by use of an inhaler or nebulizer, and formulation with an aerosolizing agent.

In a specific embodiment, it may be desirable to administer the compositions of the invention locally to a specific area of the body; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The present invention also provides pharmaceutical compositions. Such compositions comprise a therapeutically effective amount of a compound capable of regulating N-cadherin activity or N-cadherin expression and a pharmaceutically acceptable carrier. In a specific embodiment, the term “pharmaceutically acceptable” means approved by a regulatory agency of the Federal or a state government or listed in the U.S. Pharmacopeia or other Generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic compound, preferably in purified form, together with a suitable amount of carrier so as to provide the form for proper administration to the patient. The formulation should suit the mode of administration.

The amount of the compound of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose response curves derived from in vitro or animal model test systems.

The invention also provides a pharmaceutical pack or kit comprising one or more containers filled with one or more of the ingredients of the pharmaceutical compositions of the invention, optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLE

### Exogenous Expression of N-Cadherin in Breast Cancer Cells Induces Cell Migration, Invasion and Metastasis

The data presented below demonstrates that increased expression of N-cadherin in cancer cells increase cell migration, invasion and metastatic potential of cells. In addition, the expression of N-cadherin is shown to sensitize cells to FGF-2 mediated signal transduction and induces the expression of MMP-9.

### 6.1. Materials and Methods

### 6.1.1. Cell Lines

The MCF-7 and MDA-MB-435 breast cancer cell lines and endothelial HUVEC cells were obtained from the American Type Culture Collection. Mouse L-cells or L-cells transfected with mouse E-cadherin or mouse N-cadherin were provided by Dr. David Colman (Mount Sinai-NYU School of Medicine, New York). Cells were routinely cultured in DME supplemented with 10% FBS at 37° C. in a humidified 5% CO₂ atmosphere. HUVEC cells were grown in 199 media that included 10% FBS, 0.1 mg/mL heparin and 10 ng/mL FGF-2. All media and serum were from GIBCO BRL.

### 6.1.2. Reagents

Estrogen pellets (17 B-estradiol, 1.7 mg/pellet, 60-d release) were purchased from Innovative Research of America. Collagen and Matrigel were purchased from Collaborative Biomedical products. FGF-2 was purchased from Pepro Tech. Insulin, gelatin, heparin, and crystal violet were obtained from Sigma Chemical Co. Biocoat cell culture inserts (24-well) (Boyden chambers) were obtained from Becton Dickinson & Co.

### 6.1.3. Antibodies

Monoclonal anti-human E-cadherin and N-cadherin were purchased from Zymed Labs. Polyclonal antibodies directed to the ECl domain of N-cadherin were a gift from Dr. David Colman (Mount Sinai-NYU School of Medicine, New York). Ascitis-derived monoclonal anti-N-cadherin antibody was purchased from Sigma Chemical Co. and affinity-purified on 1gG columns (Pierce Chemical Co.) as indicated by the manufacturer protocol. Affinity-purified IgG₁ immunoglobulins were obtained from Zymed Labs. Monoclonal anticytokeratin (CAM 5.2) either uncoupled or directly coupled to FITC was acquired from Becton Dickinson & Co. Polyclonal anti-actin antibodies were obtained from Sigma Chemical Co. Fast DiI and Fast DiO, FITC, and rhodamine-conjugated secondary antibodies were obtained from Molecular Probes, Inc.

### 6.1.4. Constructs and Transfections

The cDNA clone for human N-cadherin (pcDNAhN-cad) (Reid and Hemperly, 1990), in the vector pcDNA-neo (Invitrogen Corp.), obtained from Dr. John Hemperly (Becton Dickinson & Co), was transfected into MCF-7 cells using Lipofectamine (GIBCO BRL). Several stable clones as well as a population of stable transfectants (mass cultures) were selected by the addition of (G418) at a concentration of 1-2 mg/mL. Clonal and mass transfectants with empty vector (pcDNA-neo) were also selected with G418. To ensure clonality of the selected clones, subsequent subcloning was performed by limiting dilution.

### 6.1.5. SDS-Page and Immunostaining N-Cadherin Transfectants

N-cadherin-transfected cells were extracted in solubilization buffer (50 mM Tris-HCl, pH 7.5, 150 mM NaCl, 0.5 mM MgCl₂, 0.2 mM EGTA, 1% Triton X-100) that included 1 mM PMSF and 10 μg/mL aprotinin and leupeptin. 20 μg of soluble protein, as determined by the BCA method (Pierce Chemical Co.), was mixed with sample buffer, boiled for 5 min, loaded on 7.5% SDS-polyacrylamide gels, and transferred onto Immobilon membranes (Millipore). Blots were probed with antibodies to N-cadherin or E-cadherin and developed with chemiluminescence (Amersham Pharmacia Biotech). The subcellular localization of N-cadherin and E-cadherin in transfected MCF-7 cells was determined by immunofluorescence using antibodies to—and E-cadherin, as previously described (Hazan R B et al., 1997, Cell Adhes. Commun 4:399-411).

### 6.1.6. Coaggregation Assays

Single cell suspensions of L-cells, L-E, L-N, MCF-7, and N-cadherin-transfected MCF-7 (N-cad-5) were prepared as described (Hazan R B et al., 1997, Cell Adhes. Commun 4:399-411), and were labeled with the fluorescent lipophilic dye Fast diI or Fast diO (Molecular Probes, Inc.). Cells (1.5×10⁵) of each type were mixed and aggregated for 20 min at 37° C. with or without 1 mM CaCl₂, as previously described (Hazan R B et al., 1997, Cell Adhes. Commun 4:399-411). Multiple aliquots from each aggregation experiment were viewed, counted, and photographed under a fluorescence microscope at a magnification of 20.

### 6.1.7. Adhesion to HUVEC Monolayers

HUVEC cells (10⁵) were plated on 1% gelatin-coated glass cover slips and allowed to form a monolayer by overnight incubation. Single cell suspensions from either Neo-5, N-cad-5, and N-cad-17 were labeled with Fast diO, and 10⁵ cells were plated over the HUVEC monolayers in 0.5 mL DME, 0.1% BSA, 1 mM CaCl₂ in a 12-well plate in triplicate wells for 6 h at 37° C. in a humidified 5% CO₂ atmosphere. Unattached cells were washed out by rinsing the cells three times with PBS. Attached cells were fixed in 3.7% paraformaldehyde for 30 min, washed, and visualized under a fluorescent microscope. Each panel is representative of triplicate experiments.

### 6.1.8. Cell Migration and Invasion through Matrigel

The ability of cells to migrate through control (migration) or invade through Matrigel-coated filters (invasion) was measured in a Boyden chamber, 8-micron Transwell filters, which were coated with or without 50 μg Matrigel (Becton Dickinson & Co.), were used according to standard protocols (Albini A et al., 1987, Cancer Res 47:3239-3245). Fibroblast conditioned medium, which was obtained by a 24-h incubation of NTH-3T3 cells with 50 μg/mL ascorbic acid in serum-free DME media, was placed in the lower chamber as a chemoattractant. Single cell suspensions of control or N-cadherin transfectants, incubated overnight in medium alone or in media containing FGF-2 (10 ng/mL) and heparin (0.1 mg/ml), were obtained by treatment with PBS containing 5 mM EDTA. Cells were washed and placed at 10⁵ cells per well into the upper chamber in 0.5 ml DME, 0.1% BSA in the presence or absence of FGF-2 (10 ng/ml) and heparin (0.1 mg/ml) for the indicated times. Cells that had not penetrated the filter were wiped out with cotton swabs, and cells that had migrated to the lower surface of the filter were stained with 0.5% crystal violet, examined by bright field microscopy, and photographed. Values for invasion were expressed as the average number of migrated cells bound per microscopic field over four fields per assay and expressed as averages for triplicate experiments.

### 6.1.9. Substrate Gel Electrophoresis (Zymography)

Secreted metalloproteinases were detected and characterized by zymography (Nakajima I et al., 1995, Br. J. Cancer. 71:1039-1045). Conditioned media were obtained by a 30-h incubation of N-cadherin and control transfected cells, which were treated overnight with or without FGF-2 (10 ng/ml) and heparin (0.1 mg/ml) in serum-free media. Conditioned media (20 μl) were loaded on 8% SDS PAGE gels that had been copolymerized with 1 mg/ml gelatin. Electrophoresis was performed under nonreducing conditions at 100 V for 2 h at 4° C. Gels were washed once for 30 min in 2.5% Triton X-100 to remove SDS, and were incubated in collagenase buffer (100 mM Tris-HCl pH 8.0, 5 mM CaCl₂, 0.02% NaN₃) for 40 h at 37° C. Gels were stained with 0.5% Coomassie blue in 30% methanol/10% acetic acid for 30 min at room temperature and destained in 30% methanol/10% acetic acid three times for 15 min. The presence of metalloproteinases was indicated by an unstained proteolytic zone in the substrate. Both active forms and pro-enzymes are revealed by this technique as the exposure of pro-MMPs to SDS during SDS-PAGE leads to activation without proteolytic cleavage. Microdissection and PCR tissue sections (4 μm) from metastatic livers and primary tumors of mice injected with either control (MCF-7) or N-cadherin-transfected cells (N-cad-5 and N-cad-17), were deparaffinized as described (Greer C E et al., 1991, Am. J. Clin. Pathol 95:117-124) and stained in 2% hematoxylin solution for 1 min. Tumor cells were microdissected from lesions in the liver under a microdissecting scope. Microdissected cells were digested overnight at 55° C. with 20 μl proteinase K in digestion buffer (1 mg/ml in 50 mM Tris-HCl buffer, pH 8.0, with 1 mM EDTA, and 0.45% Tween 20). Proteinase K digestion was stopped by incubation at 95° C. or 15 min. Cell debris was pelleted out by a centrifugation at 12,000 rpm for 10 min. 2 μl of the resultant crude DNA (supernatant) was used for PCR amplification using the T7 primer that hybridized near the cloning site in pcDNA neo and a downstream primer from human N-cadherin cDNA (5′ CACTG-TAAACATCAACAGTGAAATCC 3′). Southern blotting was performed using a nick-translated 32P-dCTP-labeled human N-cadherin cDNA probe.

### 6. 1.10 Nude Mice Experiments

Female athymic nude mice (Taconic Farms) at 6-8 wk of age, preimplanted subcutaneously with 1.7 mg of 17 B-estradiol pellets (60-d release; Innovative Research of America), were simultaneously injected bilaterally into the mammary fat pads with 10⁷ of either parental MCF-7 cells, one neomycin-resistant clone (Neo-5), or N-cadherin-transfected clones (N-cad-5 and N-cad-17, N-cad-8 and N-cad-15) as indicated. Mice (8-10 wk after injection) were killed, and the liver, lung, lymph nodes, pancreas, kidney, spleen, omentum, brain, heart, bone, and spinal skeletal muscle were removed. The organs were fixed in 10% buffered-formalin, paraffin-embedded, and sectioned at 4 μm. The primary tumors in the mammary fat pads were also removed and weighed.

### 6.1.11. Hematoxylin-Eosin (H&E) Staining and Histoimmunochemistry

20-50 sections from each organ were deparafinized and stained by H&E. Sections positive for metastases were antigen-retrieved by microwaving in citrate buffer (10 mM citric acid, pH 6.0), blocked in 5% horse serum, and stained for 2 h at room temperature using either of the following antibodies: monoclonal anticytokeratin (CAM 5.2) at a 1:2 dilution either uncoupled or coupled to FITC; monoclonal anti-E-cadherin or N-cadherin at 10 μg/ml; and polyclonal anti-actin antibodies at 1:100 dilution. Secondary detection was applied using secondary horse biotinylated anti-mouse antibodies (Vector Labs) at a 1:200 dilution followed by streptavidin-HRP (Zymed Labs) according to the manufacturer's protocol. Sections were developed in DAB solution, counterstained by H&E, and photographed under a microscope. Sections incubated with actin antibodies were incubated with anti-rabbit antibodies coupled to rhodamine, and those incubated with FITC-conjugated anti-cytokeratin antibodies were washed after the first incubation and mounted for fluorescent microscopy.

### 6.1.12. Map Kinase is Involved in N-Cadherin-Enhanced FGF-2 Stimulated MMP-9 Production

The ERK/MAPK pathway has been shown to be involved in the regulation of MMP gene transcription (66) and to affect the expression of MMP-9 (67-69). To determine whether N-cadherin/FGF-2-stimulated expression of MMP-9 is caused by increased MAPK activity, levels of MAPK activation in N-cadherin-expressing MCF-7 cells (N-cad-5) were compared to those found in control MCF-7 cells (Neo-mass) in response to FGF-2. Serum-starved control and N-cad-5 cells were treated with 10 ng/mL FGF-2 for 10 min and 30 μg of cell lysate were subjected to SDS-PAGE and immunobloting with either anti-phospho-MAPK (P-MAPK) or anti-total MAPK (T-MAPK).

### 6.2.13. Immunohistochemistry

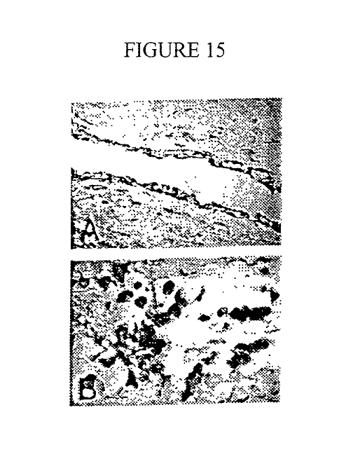

Antibodies to human E- and N-cadherin that stain frozen and formalin fixed, paraffin-embedded sections (FIG. 15) are commercially available (e.g. Zymed). Monoclonal antibodies to MMP-9 that react with both frozen and paraffin-embedded sections may be obtained (e.g. from Neo markers)(FIG. 15D).

Immunostaining of paraffin embedded breast tumor specimens may be preformed using an avidin-peroxidase method. Paraffin blocks may be sectioned at a thickness of 4 microns with sections mounted on charged slides and stored at 4° C. until use. Sections may be deparafinized and antigen retrieved by microwaving in citrate buffer, blocked in 5% horse serum, and stained for 2 hours at room temperature using the following antibodies: E-cadherin (e.g. Zymed), and human H-cadherin (e.g. Zymed), MMP-9 (e.g. Neo markers). secondary detection may be done using horse biotinylated anti-mouse antibodies (e.g. Vector Labs) at a 1:200 dilution followed by streptavidin-horseradish peroxidase (e.g. Zymed) according to the manufacturer's protocol. Sections may be developed in DAB solution and counterstained with hematoxylin.

Frozen tumor tissues have been embedded in optimal cutting temperature (OCT) compound (e.g. Miles Laboratory), snap frozen by dry ice/acetone and stored at −70° C. Frozen sections may be cut on a cryostat at a thickness of 6 microns, mounted on albumin coated slides and air dried. These sections may be stored at −70° C. until used. Breast tumors may be stained using the avidin-biotin peroxidase method. Frozen sections may be fixed with acetone for 10 minutes at −70° C. and air-dried. Endogenous peroxidase activity may be blocked by immersing the sections in methanol with 0.3% hydrogen peroxide for 30 minutes and washed in TBS (20 mM Tris pH 7.4), 150 mM NaCl) with 1 mM CaCl₂. Nonspecific binding may be blocked by incubating the sections in TBS, 3% normal horse serum, 2% skim milk, 0.1% triton X-100, 1 mM CaCl₂ for 30 minutes at 25° C. All subsequent incubations may be done in this buffer. The sections may then be incubated in a moist chamber for 24 hours at 4° C. with antibodies to E-cadherin (e.g. Zymed); human N-cadherin (e.g. Zymed); and to MMP-9 (e.g. Neo markers). Sections may then be washed three times and incubated for 30 minutes at 25° C. with biotinylated anti-mouse immunoglobulins (e.g. Vector Labs). The sections may then be incubated with ABC reagent (e.g. Vectastain ABC kit, Vector Labs) for 30 minutes at 25° C. and peroxidase stained for 4 minutes in 40 mg 3,3 diaminobenzidine tetrahydrachloide in 200 mL of 20 mM Tris pH 7.4 supplemented with 40 μL 30% hydrogen peroxide.

For both paraffin and frozen sections, the location of staining, intensity, and estimated percentage of cells exhibiting staining for each marker preferably uses the standard modified Bloom Richardson criteria, grading of intraductal carcinoma using both pattern and nuclear size determination utilizing the TNM classifications scheme. Correlation of reactivity of tumors to these molecules to clinicopathological factors may be assessed by statistical analysis using the chi-squared test.

### 6.2. Results

### 6.2.1. Characterization of the Transfectants Expressing N-Cadherin

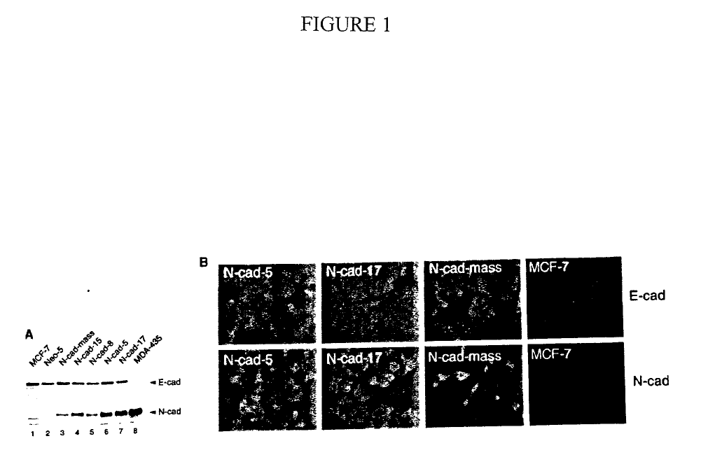

MCF-7 cells were transfected either with a vector containing the human N-cadherin cDNA (Reid R A et al., 1990, Nucleic Acids Res 18:5896) (N-cad cells) or with the vector alone (Neo-cells). Mass cultures of G418-selected stable transfectants as well as several clonal cell lines were obtained. The expression of N-cadherin in the cells was evaluated by Western blot (FIG. 1A) and immunofluorescent staining (FIG. 1B) using a human N-cadherin-specific mAb. This antibody detected a band of 130 kD in extracts of N-cad cells (FIG. 1A, bottom panel, lanes 3-7) and in MDA-MB-435 breast cancer cells (FIG. 1A, bottom panel, lane 8), which are known to express high levels of N-cadherin (Hazan R B et al., 1997, Cell Adhes. Commun 4:399-411), but not in parental MCF-7 or a clone of vector-transfected cells (Neo-5), (FIG. 1A, bottom panel, lanes 1 and 2, respectively). The level of N-cadherin in N-cad-mass cells was significantly lower than that in N-cad-5 or N-cad-17 cells (FIG. 1A, bottom panel, lane 3). Of note, E-cadherin expression was not affected by the presence of N-cadherin (FIG. 1A, top panel, lanes 1-7). Both E- and N-cadherin expression were readily detectable by immunostaining with specific mAbs, primarily in areas of cell-cell contacts (FIG. 1B, top and bottom, respectively). Expression of N-cadherin in N-cad-mass cells was heterogeneous, with only 20% positive cells (FIG. 1B, bottom panel). The expression of N-cadherin in MCF-7 cells did not alter their epithelial phenotype (FIG. 1B), and did not induce changes in either cytokeratin or vimentin levels.

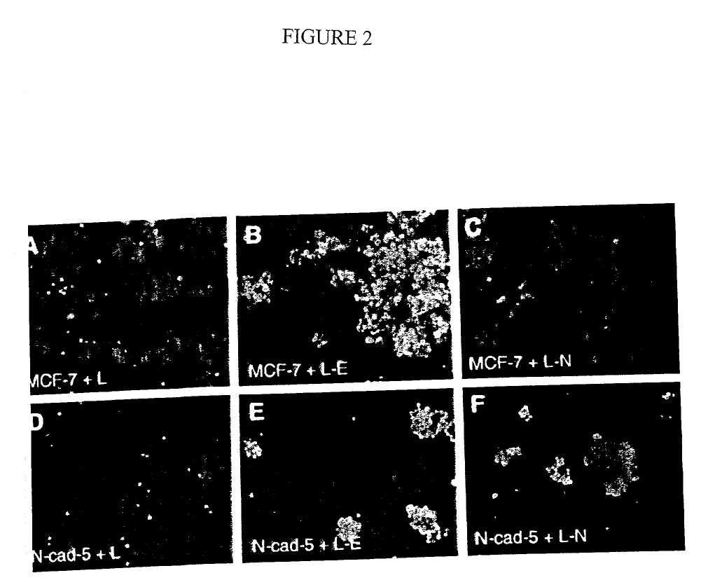

To determine whether the transfected N-cadherin was functionally active in adhesion and whether it interfered with the adhesive function of E-cadherin, parental MCF-7 cells or the N-cad-5 clone, labeled with Fast diO, were mixed and coaggregated either with parental L-cells or with L-cells transfected with either mouse E- (L-E cells) or N-cadherin (L-N cells), which were labeled with Fast diI (FIG. 2). N-Cad-5 cells formed large coaggregates with both L-E and L-N cells (FIG. 2, E and F). In contrast, MCF-7 cells aggregated with L-E but not with L-N cells (FIG. 2, B and C, respectively). Neither N-cad-5 nor MCF-7 cells aggregated with untransfected L-cells (FIG. 2, A and D), and no aggregation occurred in calcium-free conditions (data not shown). These data demonstrate that both endogenous E- and transfected N-cadherin present on the surface of MCF-7 cells are capable of mediating calcium-dependent, homotypic cellular interactions.

### 6.2.2. N-Cadherin Stimulates MCF-7 Tumor Cell Migration and Invasion

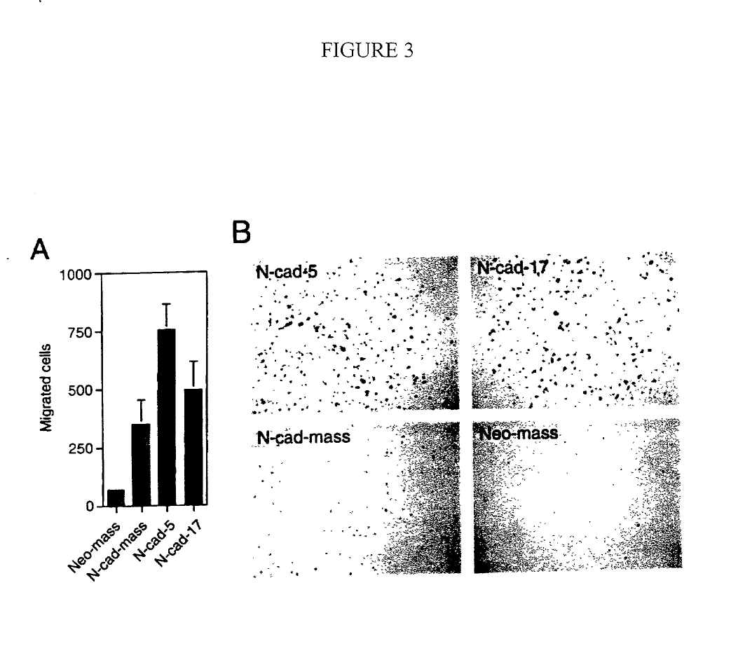

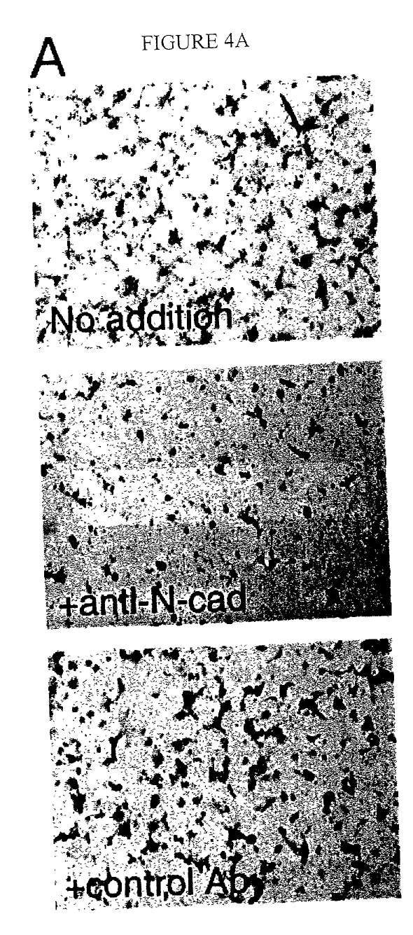

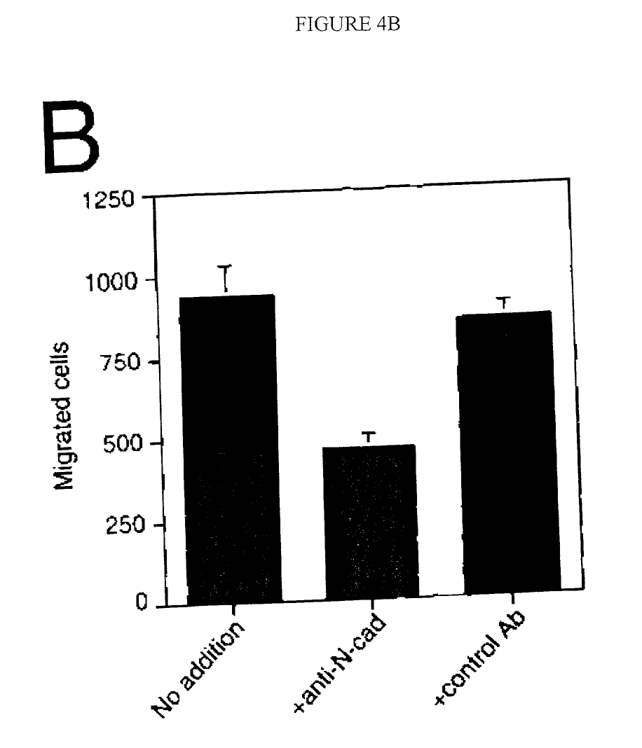

Since N-cadherin has been shown to mediate the neurite guidance and cell migration of retinal neurons (Bixby J L et al., 1990, J. Cell Biol 110:1253-1260; Doherty P et al., 1991, Neuron 6:247-258), the ability of the expression of N-cadherin by MCF-7 cells to influence cell motility was examined. The ability of cells to migrate through uncoated porous filters in response to a chemotactic stimulus was examined in a Transwell migration assay (FIG. 3). The two clones with the highest N-cadherin levels (clones 5 and 17) produced the highest numbers of migrating cells, but even the N-cad-mass cells, with only approximately one fifth of the cells expressing N-cadherin, showed a strong increase in migration over the Neo-mass control cells (FIG. 3A). Similar results were obtained when the different cell types were tested for their ability to invade through Matrigel-coated filters; only N-cadherin-expressing cells invaded into Matrigel, and the two clones with the highest expression of N-cadherin exhibited the highest number of invading cells (FIG. 3B). Furthermore, treatment of N-cad-5 cells with a purified preparation of antibodies to the NH₂-terminal domain of human N-cadherin (A-CAM) resulted in reduction of cell migration by 50% (FIG. 4), whereas isotype-matched immunoglobulins had a negligible effect on the motility of these cells in vitro. This antibody was shown to disrupt adherens junctions in cultured lens cells (Volk T et al., 1984, EMBO J 3:2249-2260; Volk T et al., 1990, Dev. Biol 139:314-326) and to inhibit cell-cell aggregation in N-cadherin-expressing breast carcinoma cells but not in E-cadherin-expressing cells (Hazan R B et al., 1997, Cell Adhes. Commun 4:399-411). These results confirm a direct involvement of N-cadherin in the acquisition of a migratory pheonotype.

### 6.2.3. Metastasis of N-Cadherin MCF-7 Transfectants in Nude Mice

The potential of N-cadherin-transfected MCF-7 cells to metastasize in vivo was tested. In several previous reports, MCF-7 cells displayed weak to undetectable levels of metastasis in this model (Clarke R et al., 1993, Breast Cancer Res. Treat 24:227-239;Kern F et al., 1994, Breast Cancer Res. Treat 31:153-165; Price J E, 1996, Breast Cancer Res Treat 39:93-102). Four independent clones expressing the highest levels of N-cadherin (N-cad-5, N-cad-17, N-cad-8, and N-cad-15), a vector transfected clone (Neo-5), and parental MCF-7 cells were injected into the mammary fat pads of estrogen-treated female nude mice. A total of five to six mice were injected per cell line. 8-10 wk after injection, the primary tumors and distal organs including the liver, lung, pancreas, kidney, brain, lymph nodes, heart, spleen, omentum, salivary glands, and skeletal muscle were resected, fixed, paraffin-embedded, sectioned, and stained either with H&E or with an anti-human cytokeratin-specific antibody (Moll R et al., 1982, Cell 31:11-24).

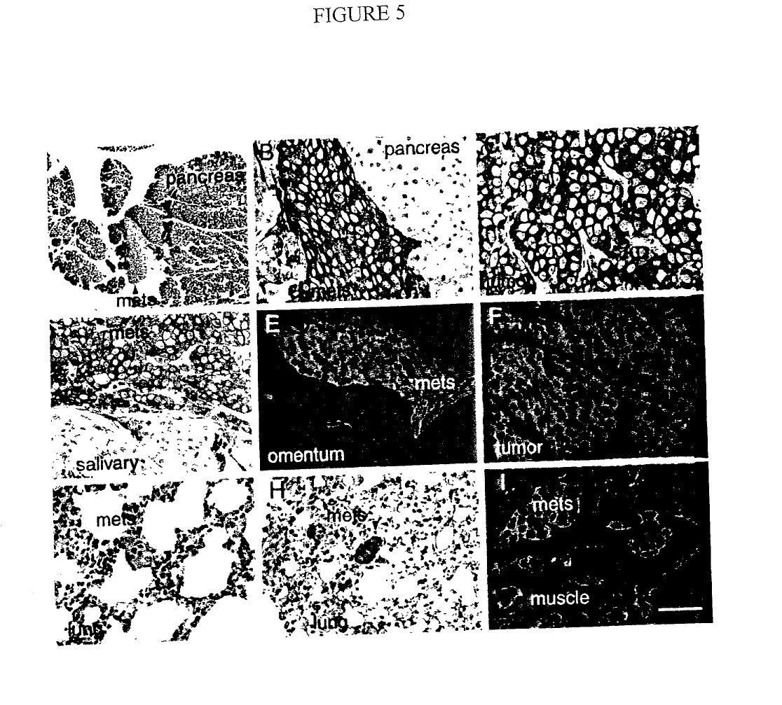

Of a total of 20 mice injected with N-cadherin-expressing cell lines, 13 mice (65%) had primary tumors. Similarly, 10 out of 12 mice (83%) injected with control MCF-7 cells developed primary tumors. N-cadherin-expressing tumors grew slower on average, reaching a weight of ˜30% that of control MCF-7 or Neo-5 tumors (Table I). This is consistent with the slower growth rate of the N-cadherin-transfected cells in vitro (data not shown). Most (77%) of the 13 mice with N-cadherin-expressing primary tumors developed metastases in multiple sites, whereas none of the control mice bearing larger tumors had detectable metastases (Table I). Staining with H&E strongly suggested the presence of metastatic cells in the different organs (for examples see FIG. 5, A-G). The human epithelial origin of these cells was confirmed by staining with an anti-cytokeratin antibody either directly coupled to FITC (FIG. 5, E, F, and I) or followed by secondary HRP detection (FIG. 5, B-D and H). This antibody reacted only with human and not with mouse cytokeratin. The sections of the primary tumors stained equally well, regardless of the method of detection, (peroxidase or FITC) (FIG. 5, C and F). FIG. 5 shows a representative panel of sections from different organs in which metastases were found. In most organs (pancreas, salivary gland, omentum, and muscle, FIG. 5, B, D, E, and I, respectively), large areas of metastatic growth were found, but micrometastases shown in a lung section (FIG. 5, G and H) were also present. In a comparable analysis of tissue sections from mice injected with control MCF-7 or Neo-5 cells, no cytokeratin-reactive cells were ever found in the pancreas, lymph nodes, salivary gland, omentum, liver, lung, and skeletal muscle (Table I), or in other tissues (data not shown). As indicated in Table I, whereas the metastases produced by other clones were more random, the two clones with the highest level of N-cadherin expression produced liver metastases in almost all injected mice.

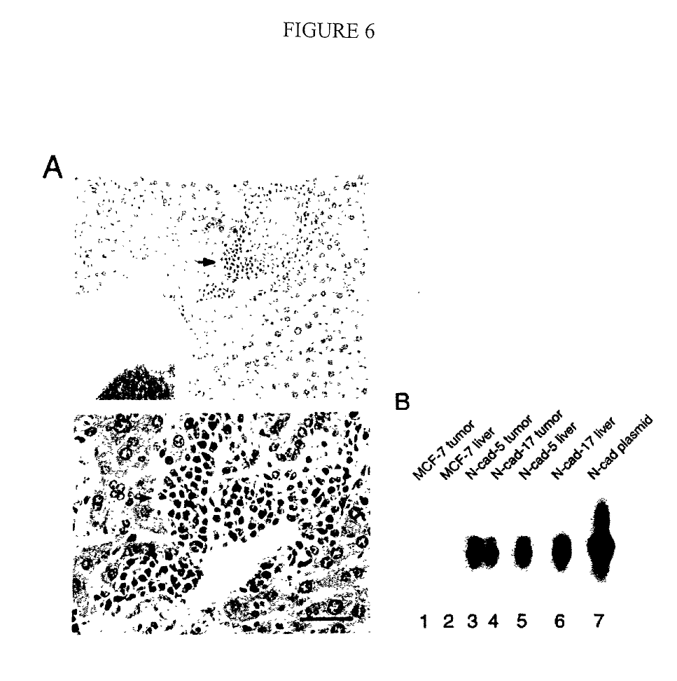

To verify the presence of the transfected N-cadherin in metastases, DNA was extracted from tumor cells that were microdissected from areas of liver sections deemed to be positive for metastasis by H&E staining (FIG. 6A, top and bottom show a section from the liver of an N-cad-17-inoculated mouse) and used in a PCR reaction designed to detect the presence of the transgene (see Materials and Methods). Southern blotting of the reaction products using the human N-cadherin cDNA as a probe confirmed the specificity of the detected band as the transfected N-cadherin (FIG. 6B, lane7). DNA isolated from an MCF-7 primary-tumor, or from a liver of a MCF-7 tumor-bearing mouse did not yield an amplification product (FIG. 6B, lanes 1 and 2). In contrast, DNA isolated from primary tumors and livers of N-cad-5- and N-cad-17-inoculated mice were positive in the PCR reaction (FIG. 6B, lanes 3-6).

TABLE I
N-Cadherin Induces Breast Cancer Metastasis in Nude Mice

| | | | Metagstatic Organs | | | | | | |
|---|---|---|---|---|---|---|---|---|---|
| | Primary | | | | Salivary | | | | Lymph |
| Cell lines | tumor g | n | Liver | Pancreas | gland | Omentum | Lung | Muscle | nodes |
| N-cad-5 | 0.35 ± 0.1 | 4 | 3/3 | 0/2 | 3/4 | 2/3 | 0/3 | 0/3 | 2/4 |
| N-cad-17 | 0.27 ± 0.09 | 3 | 2/3 | 3/3 | 0/2 | 0/2 | 0/2 | 0/3 | ND |
| N-cad-8 | 0.23 ± 0.08 | 3 | 0/3 | 0/3 | 0/3 | 0/3 | 2/3 | 0/3 | 0/3 |
| N-cad-15 | 0.25 ± 0.1 | 3 | 0/3 | 0/3 | 0/3 | 0/3 | 2/3 | 2/3 | 0/3 |
| Neo-5 | 1.20 ± 0.2 | 5 | 0/5 | 0/5 | 0/5 | 0/5 | 0/5 | 0/5 | 0/5 |
| MCF-7 | 1.20 ± 0.3 | 5 | 0/5 | 0/5 | 0/5 | 0/5 | 0/5 | 0/5 | 0/5 |

Breast cancer metastasis in nude mice injected with either MCF-7, Neo-5 control cells (Neo) or N-cadherin-transfected cell lines (N-cad-5 and N-cad-17, N-cad-8 and N-cad-15) was monitored by H&F and staining for human cytokeratin in at least 20 sections of each indicated organ. The number of mice positive for metastasis over the total number of screened mice is indicated. The number of mice that developed primary tumors at the mammary fat pads (n) and their mass is indicated by average weight in grams (g)±SD. Several additional organs such as the kidney, spleen, bone, brain and skeletal muscle were tested and found negative. ND, not determined.

### 6.2.4. Coexpression of Cadherins E and N in Metastatic Lesions

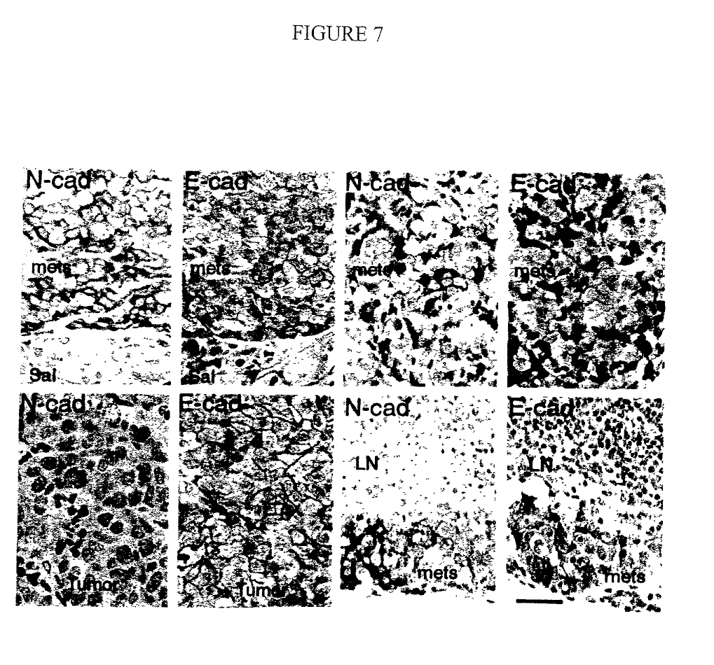

The preceding data show that N-cadherin transfection has a striking effect on the metastasis of MCF-7 breast tumor cells. It remained to be determined whether metastatic cells in vivo retained—and E-cadherin expression. Staining of metastatic lesions in the salivary gland, pancreas (FIG. 7, top) and axillary lymph nodes (bottom right panels) as well as in the lumbar muscle (data not shown), with anti-human E- or N-cadherin antibodies revealed that both cadherins were present in areas of contact between tumor cells. Note the massive infiltration of tumor cells into the salivary gland and pancreas but only marginal invasion of the lymph nodes. In contrast, N-cadherin staining was not detected in primary tumors produced by the non-metastatic, non-N-cadherin-expressing MCF-7 cells, which continued to express E-cadherin (FIG. 7, bottom left). Since N-cadherin expression was conserved during the metastatic progression of transfected MCF-7 tumor cells, it must play a dominant role over E-cadherin in inducing this phenotype.

### 6.2.5. FGF-2 Enhances Migration of N-Cadherin-Expressing Cells, up Modulates Their MMP-9 Production and Increases Matrigel Invasion

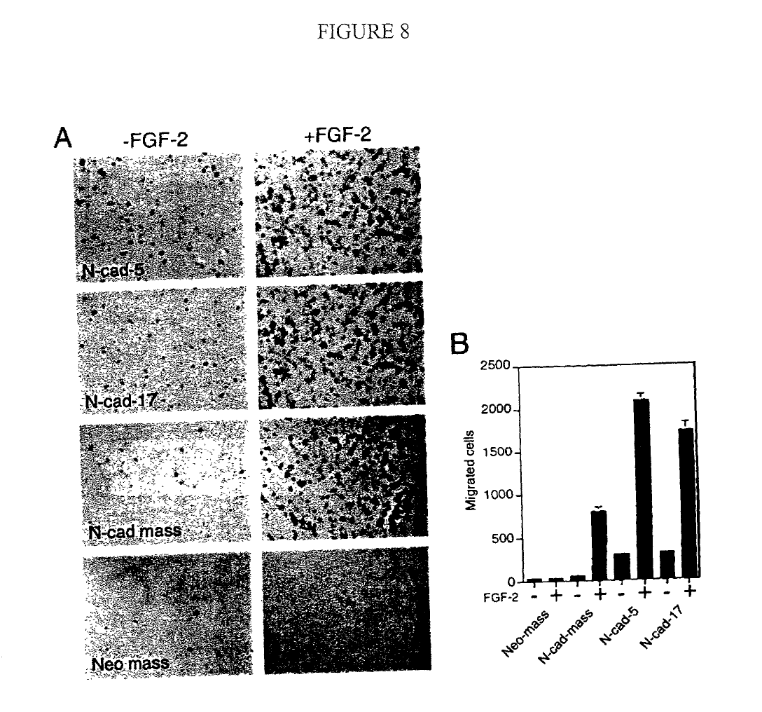

N-cadherin has been postulated to promote neurite outgrowth by a mechanism that involves a cooperative interaction between N-cadherin and FGFR (Williams E J et al., 1994, Neuron 13:583-594; Doherty P et al., 1996, Mol. Cell. Neurosci 8:99-111; Saffell J L et al., 1997, Neuron 18:231-242; Lom B et al., 1998, J. Neurobiol 37:633-641). In an attempt to evaluate whether these two molecules cooperate to promote the metastasis of N-cadherin-expressing MCF-7 cells, the effect of FGF-2 on motility and invasion of control and N-cadherin transfectants was examined. Treatment with FGF-2 did not affect the motility of the Neo-mass cell line, but caused a dramatic increase in motility in all N-cadherin-expressing cell lines (FIG. 8, A and B).

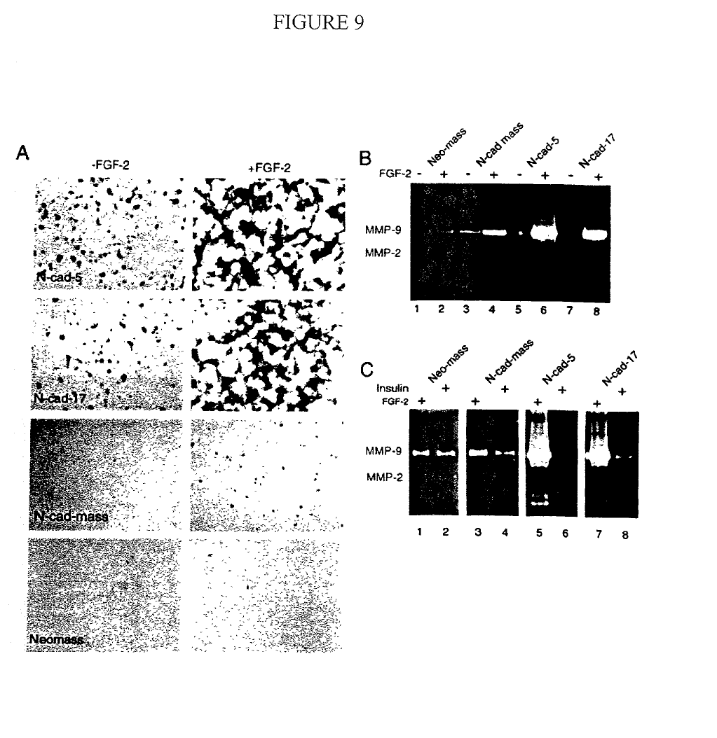

The effect of FGF-2 on the invasive activity of N-cadherin-expressing MCF-7 cells was next assessed in a Matrigel invasion assay after 5 h of incubation (FIG. 9A). The low basal invasiveness of the N-cad mass cells and the high invasiveness of the N-cad-5 and -17 clones were all further enhanced by FGF-2 treatment. In contrast, there was not effect on the Neo-mass cell invasion (FIG. 9A). It was observed that the Matrigel-invading N-cadherin-transfected MCF-7 cells appeared on the underside of the filter as cell clusters rather than individual cells. This suggests that the gain of invasive properties by N-cadherin does not involve a switch from an adhesive to a more scattered, mesenchymal phenotype usually observed with invasive carcinomas (Oka H et al., 1993, Cancer Res 53:1696-1701; Sommers C L et al., 1994, Breast Cancer Res. Treat 31:325-335; Hazan R B et al., 1997, Cell Adhes. Commun 4:399-411).

Since only N-cadherin-expressing cells responded to FGF-2 treatment with increased invasion of Matrigel, it was determined whether the response was linked to elevated levels of MMPs, which are well documented ECM-degrading enzymes and whose activity is associated with tumor invasiveness (Coussens L M et al., 1996, Chem. Biol 3:895-904). MMP activities were measured by a zymography assay of conditioned media from N-cadherin-transfected and control MCF-7 cells, untreated or treated with FGF-2 (Nakajima I et al., 1995, Br. J. Cancer. 71:1039-1045). Conditioned media collected in the absence of FGF-2 all produced readily detectable small zones of lysis corresponding to MMP-9 and barely detectable zones of lysis corresponding to MMP-2 (FIG. 9B, lanes 1, 3, 5, and 7). FGF-2 had no effect on MMP production by Neo-mass cells (FIG. 9B, lane 2). However, the same growth factor dramatically increased the expression of MMP-9 in all N-cadherin transfectants in the absence of detectable effects on MMP-2 production (FIG. 9B, lanes 4, 6, and 8). The effect of FGF-2 on MMP-9 levels was more pronounced in N-cad-5 and N-cad-17 (FIG. 9B, lanes 6 and 8) than in N-cad-mass cultures (FIG. 9B, lane 4), which is consistent with the lower levels of N-cadherin expression in the latter (FIG. 1). Treatment with insulin at a concentration that acts through the IGF-1 receptor produced no effect on MMP levels (FIG. 9C, lanes 4, 6 and 8) despite the high levels of IGF-1 receptors expressed by MCF-7 cells (Cullen K J et al., 1990, Cancer Res 50:48-53; Surmacz E et al., 1998, Breast Cancer Res. Treat 47:255-267). An additional doublet of 52/46 kD was occasionally observed in conditioned media of FGF-2-treated N-cad-5 and N-cad-17 (FIG. 9C, lanes 5 and 7), and comigrated with MMP-1 or stromelysin (Herron G S et al., 1986, J. Biol. Chem 261:2814-2818). The above results imply that N-cadherin expression sensitizes MCF-7 cells to FGF-2, and that this combined effect results in an increased cell migration, enhanced MMP-9 production, and efficient invasion of ECM proteins. Thus, the coordinated action of N-cadherin, FGF-2, and MMP-9 provides a possible mechanism by which N-cadherin my promote cellular invasion and metastasis in vivo.

### 6.2.6. N-Cadherin Promotes Adhesion to the Endothelium

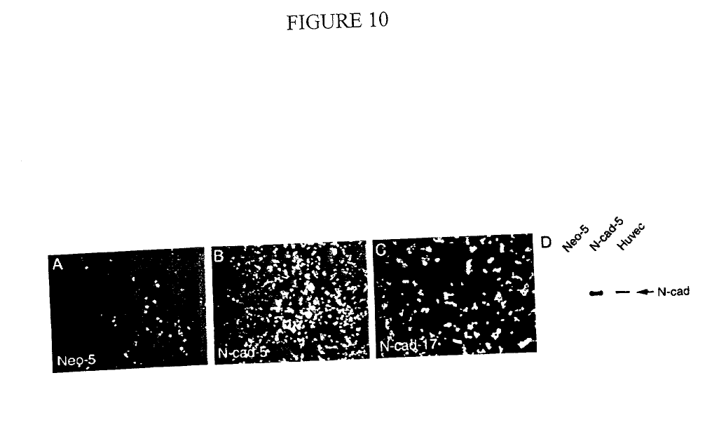

Additional factors that may critically affect tumor cell metastasis involve interactions between tumor cells and the endothelium. Since endothelial cells are known to express N-cadherin (Salomon D et al., 1992, J. Cell Sci 102:7-17), it is possible that the N-cadherin expressed on the surface of tumor cells might promote hemophilic interactions with the endothelium. The ability of control and N-cadherin-expressing MCF-7 cells to adhere to human endothelial monolayers was tested. Whereas N-cad-5 and to a lesser extent, N-cad-17 cells adhered strongly to endothelial cells (FIG. 10, B and C, respectively), control Neo-5 cells exhibited a much weaker adhesion (FIG. 10A). To control for the relative expression of N-cadherin in these cells, equal amounts of protein extracts from Neo-5 cells, N-cad-5, and HUVEC cells (FIG. 10D) were immunoblotted with anti-N-cadherin EC1 antibodies. In contrast to control Neo-5 cells, which do not express N-cadherin, HUVEC cells had detectable amounts of N-cadherin, although lesser than that found in N-cad-5 cells. Thus, the ability of N-cadherin-expressing MCF-7 cells to adhere to N-cadherin-expressing endothelial sheets may facilitate their transit through the vasculature and improve their ability to form metastasis. In support of these findings, N-cadherin has been shown to mediate the transmigration of melanoma cells through the endothelium (Sandig M et al., 1997, Cell Motil. Cytoskeleton 38:351-364; Voura E B et al., 1998, Microsc. Res. Tech 43:265-275).

### 6.2.7. N-Cadherin FGF Receptor Interaction

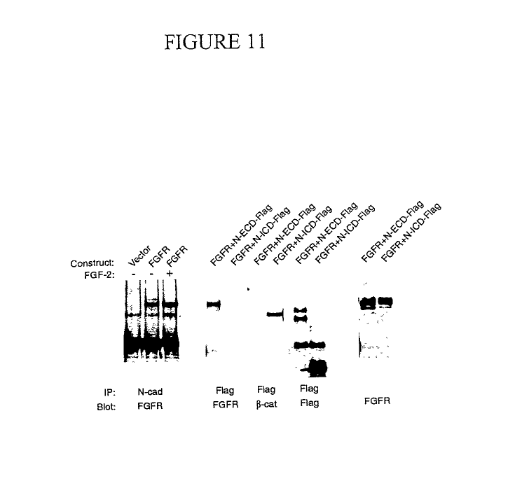

N-cadherin associates with the FGF receptor through its extracellular domain FIG. 11). 293T cells (N-cadherin positive) transfected with either FGFR (FIG. 11, lanes 2 and 3) or with vector alone (FIG. 11, lane 1) were treated with or without FGF-2 and immunoprecipitated with anti N-cadherin antibodies and probed with anti-FGFR. The specific co-immunoprecipitation of N-cadherin with FGFR does not require FGF-2 as overexpression of FGFR results in constitutive activation of the receptor.

293T cells were transfected with FGFR+Flag-tagged N-cadherin extracellular domain (N-ECD-Flag)(lane 4) or FGFR+N-cadherin intracellular domain (N-ICD-Flag)(lane 5) and immunoprecipitated with anti-Flag-tag antibodies and blotted with anti-FGFR antibodies. Note the selective co-immunoprecipitation of FGFR with the N-cadherin extracellular domain and not at all with the intracellular domain although this domain was expressed at high levels (lane 9) and was able to associate intracellularly with β-catenin as expected (lane 7).

The expression of N-ECD-Flag or N-ICD-Flag in cell lysates is shown in FIG. 11, lanes 8 (80 Kd doublet band) and 9 (30 Kd band) respectively by immunoblotting with anti-Flag-tag antibodies. The levels of FGFR in 293T co-transfected with N-ECD-Flag (FIG. 11, lane 10) or N-ICD-Flag (FIG. 11, lane 11) were identical.

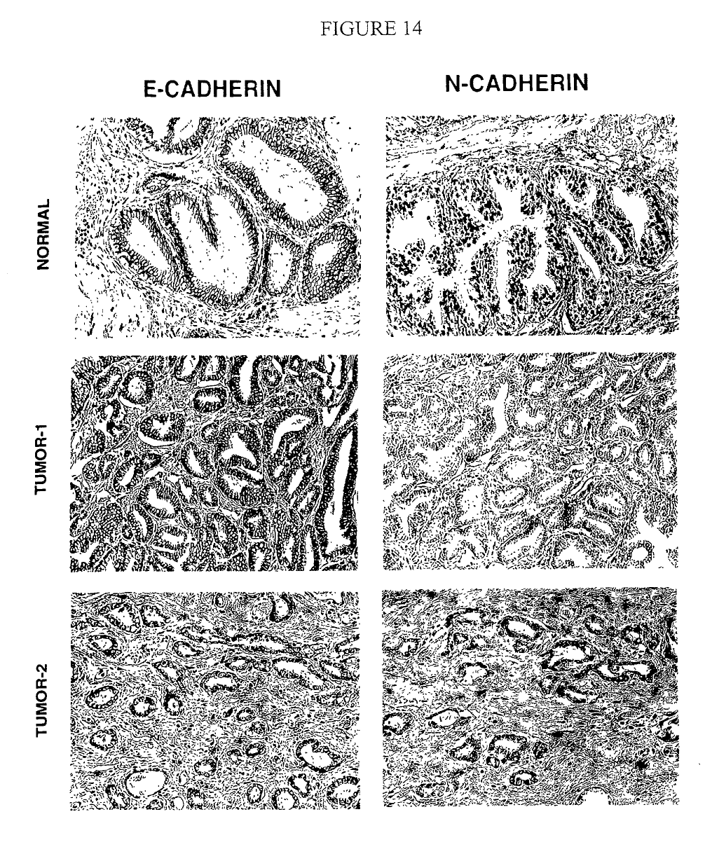

Specific co-precipitation of N-cadherin with FGFR-1 in 293T cells transiently transfected with FGFR-1 indicates that N-cadherin forms a complex with FGFR-1 (FIG. 14). Lysates from N-cadherin expressing 293T cells transfected with FGFR-1 were untreated or treated with 10 ng/mL FGF-2 and were immunoprecipitated with either control IgG (lane 1) or with FGFR-1 antibodies (Lanes 2, 3) and blotted with anti-N-cadherin antibodies.

### 6.2.8. Map Kinase is Involved in N-Cadherin-Enhanced FGF-2 Stimulated MMP-9 Production

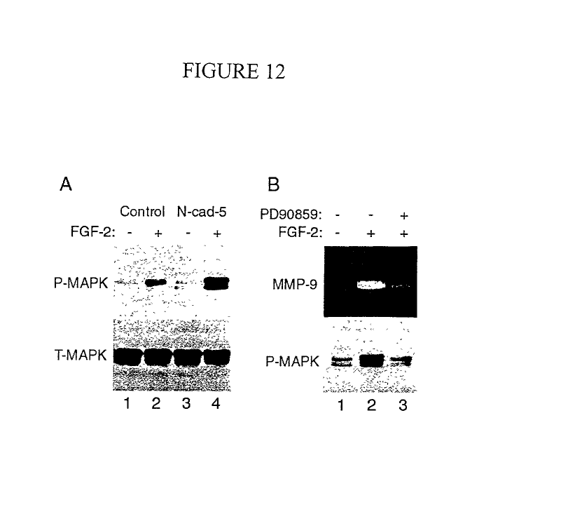

N-cadherin synergises with FGF-2 in promoting MAPK activation and MMP-9 production (FIG. 12). MCF-7 breast cancer cells expressing either control vector (control) or N-cadherin (N-cad-5) were treated with or without FGF-2 and the relative activation of MAPK was assessed by blotting of the lysates with anti phospho-MAPK (P-MAPK) (FIG. 12A, upper panel, lanes 1-4) against the total levels of MAPK in the same lysates (T-MAPK) (FIG. 12A, lower panel, lanes 1-4). A marked increased in phosphorylated MAPK in MCF-7 positive for N-cadherin (FIG. 12A, lanes 3 and 4) above the levels found in MCF-7 lacking N-cadherin (FIG. 12, lanes 1 and 2) was observed.

MCF-7 breast cancer cells expressing N-cadherin (N-cad-5) were treated with or without FGF-2 in the absence or presence of a potent inhibitor of MAPK (PD 90859) and cell lysates were blotted with anti-P-MAPK antibodies (FIG. 12B, lower panel) or assayed for MMP (Matrix Metallo Protease) production (FIG. 12B, upper panel). PD 90859 blocked effectively the phosphorylation of MAPK induced by FGF-2 (FIG. 12B, lower panel, compare lane 3 to lanes 1-2) and also blocked the production of MMP-9 that was induced by FGF-2 (FIG. 12B, upper panel, compare lane 3 to lanes 1-2).

FGF-2 stimulated a marked increase in MAPK phosphorylation in N-cad-5 as shown by the reactivity of phospho-MAPK (P-MAPK) antibodies with the MAPK/ERK isozymes (P42/P44) in total cell lysates (FIG. 12A, lanes 3,4). In contrast, control cells displayed only low levels of phosphorylation of the ERK/P44 isoform in the presence of FGF-2 (FIG. 12A, lanes 1,2). Changes in MAPK were similar in both cell lines (FIG. 12A, lanes 1-4). These results suggest that N-cadherin enhances the activations of FGFR by FGF-2, thus leading to more efficient downstream coupling to the MAPK pathway.

### 6.2.9. NIH-3T3 Cells Experiments Indicate that MAPK Induced MMP-9 Production VIA FGFR/N-Cadherin is a Generalized Mechanism

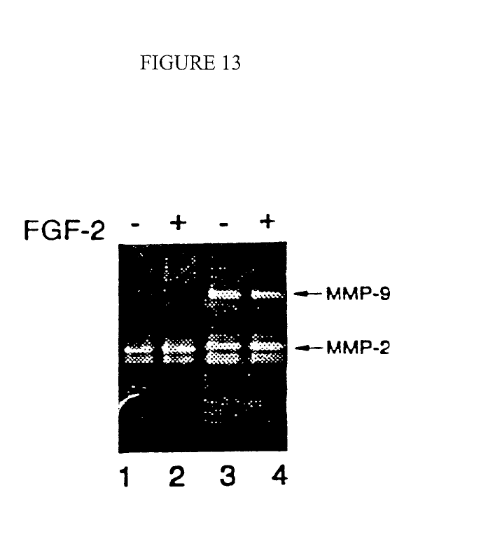

Fibroblasts (NIH-3T3) provide a different cellular context and thus rules out potential artifacts of cell type specificity. NIH-3T3 cells that express high endogenous levels of both N-cadherin and MMP-2, but not MMP-9, were transfected with control vector or FGFR-1 cDNA. Transfection of FGFR-1 induced the expression of MMP-9 in NIH-3T3 cells. MMP-9 induction was independent of FGF-2 stimulation as these cells express constitutively activated FGFR-1 due to overexpression (FIG. 13).

As depicted in FIG. 15, antibodies to human E-cadherin stain the well differentiated part of the tumor while N-cadherin antibodies stain the de-differentiated breast tumor cells well within the same tumor. (FIG. 15).

Various publications are cited in the specification, the contents of which are hereby incorporated by reference in their entireties.

What is claimed:

1. A method for identifying a compound that inhibits N-cadherin activity comprising:
(i) contacting a cell expressing N-cadherin with a test compound and measuring the level of N-cadherin activity;
(ii) in a separate experiment, contacting a cell expressing N-cadherin with the test compound vehicle control and measuring the level of N-cadherin activity, where the conditions are essentially the same as in part (i); and
(iii) comparing the level of N-cadherin activity measured in part (i) with the level of N-cadherin activity in part (ii),
wherein a decrease level of N-cadherin activity in the presence of the test compound indicates that the test compound is a N-cadherin inhibitor.

2. The method of claim 1 wherein step (i) and (ii) further comprises the addition of FGF-2.

3. The method of claim 1 or 2 wherein the activity of N-cadherin is measured using a cell aggregation assay.

4. The method of claim 1 or 2 wherein the activity of N-cadherin is measured using a cell migration assay.

5. The method of claim 1 or 2 wherein the activity of N-cadherin is measured using a cell adhesion assay.

6. The method of claim 2 wherein the activity of N-cadherin is measured by detecting the level of MMP-9 expression.

7. A method for inhibiting the metastatic potential of a cell comprising contacting said cell with an inhibitor of N-cadherin activity.

8. The method of claim 7 wherein the inhibitor inhibits the expression of N-cadherin in the cell.

9. The method of claim 8 wherein the inhibitor inhibits the transcription of N-cadherin in the cell.

10. The method of claim 8 wherein the inhibitor is an antisense or ribozyme nucleic acid molecule that blocks N-cadherin mRNA translation.

11. The method of claim 7 wherein the inhibitor prevents the binding of fibroblast growth factor receptor to N-cadherin.

12. The method of claim 7 wherein the inhibitor prevents the expression of matrix metalloprotinase-9 activity.

13. The method of claim 7 wherein the inhibitor of N-cadherin activity is an N-cadherin antibody.

14. A method for treating a proliferative disorder in a subject comprising administering to the subject a compound that modulates the synthesis, expression or activity of N-cadherin so that the symptoms of the disorder are ameliorated.

15. The method of claim 14 wherein the proliferative disorder is cancer.

16. The method of claim 14 wherein the compound inhibits the N-cadherin mediated metastatic potential of a cancer cell.

17. The method of claim 14 wherein the compound inhibits binding of fibrobalst growth factor receptor to N-cadherin.

18. A method for determining the presence of a cancer in a subject, comprising the steps of:
(i) contacting a biological sample obtained from a subject with a reagent capable of detecting the level of N-cadherin expression within the sample; and
(ii) comparing the level of N-cadherin expression in the sample with the level of N-cadherin expression in a control sample
wherein an increase in the level of N-cadherin expression detected in the sample, compared to the control sample, indicates the presence of a cancer in the subject.

19. The method of claim 18 in which expression is measured by detecting N-cadherin protein.

20. The method of claim 18 in which expression is measured by detecting N-cadherin transcripts.

21. A method for monitoring the progression of a cancer in a subject comprising:
(i) contacting a biological sample obtained from a subject at a first time point with a reagent capable of detecting the level of N-cadherin expression within the sample;
(ii) contacting a biological sample obtained from a subject at a subsequent time point with a reagent capable of detecting the presence of N-cadherin expression within the sample; and
(iii) comparing the level of N-cadherin expression present in step (i) with the level of N-cadherin present in step (ii)
wherein an increase in the level of N-cadherin detected in step (i), compared to the level of N-cadherin detected in step (ii), indicates the progression of a cancer in the subject.

22. The method of claim 21 in which expression is measured by detecting N-cadherin protein.

23. The method of claim 21 in which expression is measured by detecting N-cadherin transcripts.

24. A method for determining the presence of a cancer in a subject, comprising the steps of:
(i) contacting a biological sample obtained from a subject with a reagent capable of detecting the level of matrix metalloproteinase-9 expression within the sample; and
(ii) comparing the level of metalloproteinase-9 expression in the sample with the level of metalloproteinase-9 expression in a control sample
wherein an increase in the level of metalloproteinase-9 expression detected in the sample, compared to the control sample, indicates the presence of a cancer in the subject.

25. A method for monitoring the progression of a cancer in a subject comprising:
(i) contacting a biological sample obtained from a subject at a first time point with a reagent capable of detecting the level of metalloproteinase-9 expression within the sample;
(ii) contacting a biological sample obtained from a subject at a subsequent time point with a reagent capable of detecting the presence of metalloproteinase-9 expression within the sample; and
(iii) comparing the level of metalloproteinase-9 expression present in step (i) with the level of metalloproteinase-9 expression present in step (ii)
wherein an increase in the level of metalloproteinase-9 expression detected in step (i), compared to the level of metalloproteinase-9 expression detected in step (ii), indicates the progression of a cancer in the subject.
